(19) United States
(12) Patent Application Publication
(10) Pub. No.: US 2026/0290578 A1
(43) Pub. Date: Sep. 24, 2026

(54) SYSTEM

(71) Applicant: SoftBank Group Corp., Tokyo (JP)

(72) Inventor: Kazuki SHIMIZU, Tokyo (JP)

(73) Assignee: SoftBank Group Corp., Tokyo (JP)

(21) Appl. No.: 19/567,357

(22) Filed: Mar. 16, 2026

(30) Foreign Application Priority Data
Mar. 19, 2025 (JP) 2025-044981

(51) Int. Cl. G16H 40/67; G06F 16/25; G06F 40/40; G06N 5/04; G16H 50/30

(52) U.S. Cl. CPC G16H 40/67; G06F 16/258; G06F 40/40; G06N 5/04; G16H 50/30

(57) ABSTRACT

A system includes a processor that is configured to collect health information of a user by using a sensor device and store the health information in a database, generate a prompt for instructing a generative AI model to perform analysis in order to analyze the stored health information, input the prompt to the generative AI model, and obtain an analysis result from the generative AI model, and generate appropriate health advice for the user based on the obtained analysis result and notify a terminal of the user of the health advice.

### CROSS-REFERENCE TO RELATED APPLICATION

This application is based on and claims priority under 35 USC 119 from Japanese Patent Application No. 2025-044981 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

In recent years, various sensor devices, such as wearable devices and smartphones, have been widely used to measure and record health information of users, including heart rate, physical activity, sleep state, and other biometric parameters. Although such devices can continuously collect large amounts of health-related data, conventional health management systems generally rely on fixed rule-based analysis or simple statistical processing and therefore have difficulty providing highly personalized and context-aware health advice to individual users. As a result, the health advice presented to users tends to be generic, lacks consideration of detailed health history and lifestyle context, and does not sufficiently support concrete behavioral changes for improving long-term health outcomes.

Furthermore, even when advanced machine learning models are used, the process for effectively utilizing them is often complex and not optimized for dynamically guiding an external generative AI model by using prompts that appropriately reflect the user's current and past health conditions. Conventional systems do not fully exploit the potential of generative AI models to produce rich, natural-language analysis and recommendations that are tailored to each user's unique situation.

Accordingly, there is a need for a system capable of: (i) collecting and storing health information of a user through sensor devices; (ii) generating prompts that instruct a generative AI model to analyze the stored health information while taking into account the user's past health history; and (iii) generating, based on an obtained analysis result, appropriate health advice that includes specific action proposals for improving the user's lifestyle habits, and notifying such advice to a terminal of the user. The present invention has been made in view of these circumstances.

### SUMMARY

In order to solve the above-described problems, an aspect of the present invention provides a system comprising a processor, wherein the processor is configured to collect health information of a user by using a sensor device and store the health information in a database. The processor is further configured to generate a prompt for instructing a generative AI model to perform analysis in order to analyze the stored health information, input the prompt to the generative AI model, and obtain an analysis result from the generative AI model. The processor is also configured to generate appropriate health advice for the user based on the obtained analysis result and notify a terminal of the user of the health advice.

According to another aspect, the processor is configured to generate a prompt that instructs the generative AI model, when analyzing the health information of the user, to take past health history of the user into account as part of the prompt, thereby improving analysis accuracy by enabling the generative AI model to consider temporal trends, previous diagnoses, and historical lifestyle patterns of the user.

According to still another aspect, the processor is configured to generate the health advice based on the analysis result obtained from the generative AI model so as to include specific action proposals for improving lifestyle habits of the user, such as concrete exercise plans, sleep improvement measures, stress reduction techniques, and daily behavioral recommendations. By combining sensor-based data collection, prompt-driven analysis using a generative AI model, and generation of personalized, actionable health advice, the system according to the present invention can effectively support the user in improving health conditions and lifestyle habits in a continuous and individualized manner.

The term “processor” refers to one or more hardware-based computing elements, such as a central processing unit (CPU), microprocessor, microcontroller, or a combination thereof, that execute instructions to perform functions of the system including data collection, storage control, prompt generation, model interaction, and advice generation.

The term “sensor device” refers to any device or component, including a wearable device, smartphone, smartwatch, fitness tracker, medical sensor, or a combination thereof, that measures or detects physical, physiological, or behavioral parameters of a user and outputs corresponding digital health information.

The term “health information of a user” refers to data related to the physical, physiological, or mental condition of the user, including but not limited to heart rate, blood pressure, activity level, step count, sleep duration and quality, body temperature, weight, stress level, and other biometric or wellness-related indicators.

The term “database” refers to a structured electronic storage system, which may be implemented using one or more memory units, storage devices, or database management systems, for persistently storing and managing health information, analysis results, user history, and related data.

The term “generative AI model” refers to an artificial intelligence model, such as a large language model or other generative model, trained on data to generate outputs including natural language text or other structured information in response to input prompts, and used in the system to analyze stored health information and produce analysis results.

The term “prompt” refers to a structured input, including textual or formatted content, generated by the processor and provided to the generative AI model, the prompt specifying instructions, context, and data to guide the generative AI model in performing analysis of the user's health information.

The term “analysis result” refers to information output from the generative AI model in response to a prompt, the information including evaluations, interpretations, assessments, summaries, or inferences regarding the user's health information and related conditions.

The term “health advice” refers to information generated by the processor based on the analysis result, the information including recommendations, guidance, cautions, or suggestions related to the user's health, wellness, or lifestyle, and intended to support improvement or maintenance of the user's health condition.

The term “terminal of the user” refers to an electronic device operated or owned by the user, such as a smartphone, tablet, personal computer, or wearable device with display or notification capability, that receives notifications from the system and presents health advice to the user.

The term “past health history of the user” refers to accumulated historical health information of the user stored in the database, including past measurements, past analysis results, prior health advice, known conditions, and recorded lifestyle patterns over time.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

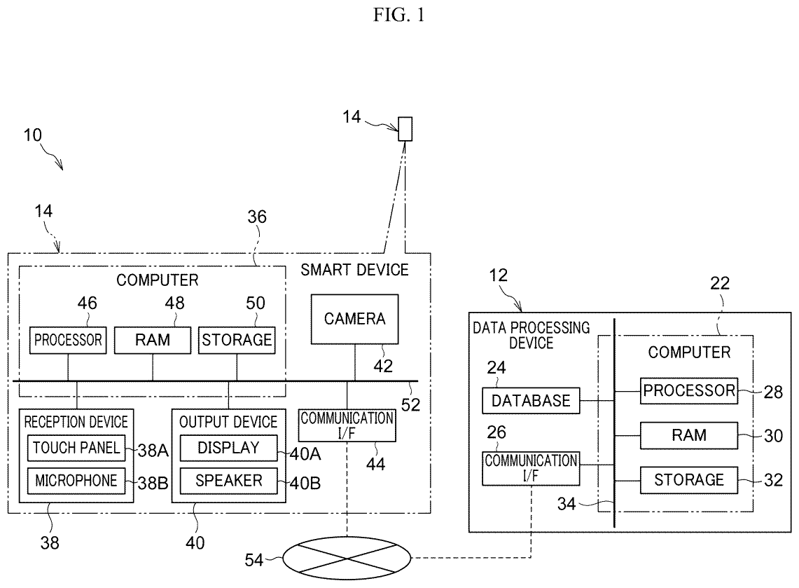

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

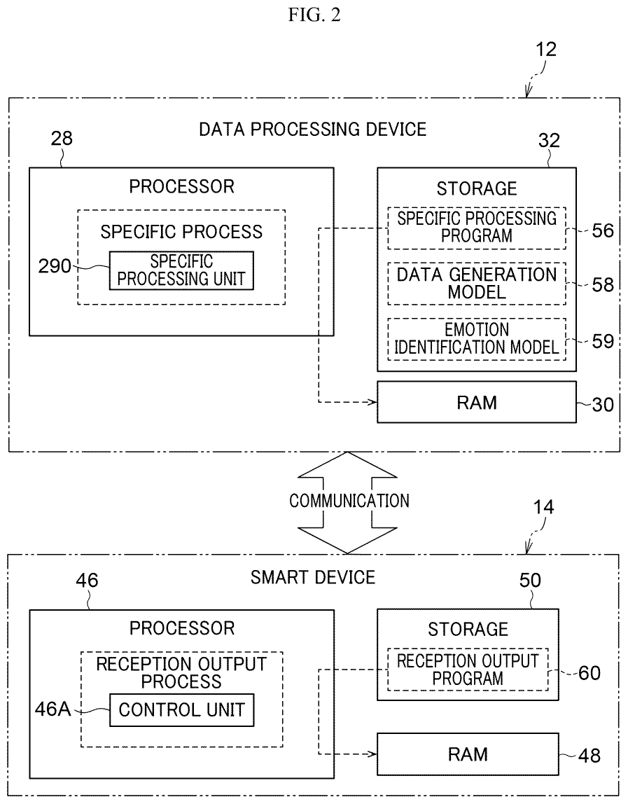

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented health advice systems typically rely on static rule sets or simple statistical thresholds applied directly to raw sensor readings. Such systems suffer from several technical problems. First, they treat heterogeneous time-series data from multiple devices and manual inputs in an ad hoc manner, without a unified data aggregation and preprocessing layer. As a result, missing values, outliers, and inconsistent sampling intervals are handled poorly, which degrades the quality of any downstream computational analysis and causes unstable behavior of the overall system. Second, existing systems generally invoke machine-learning models or generative AI models in isolation, without explicitly structuring intermediate analysis results into a machine-readable and model-oriented input representation. This leads to inefficient use of computing resources, since generative models are forced to infer basic patterns directly from noisy raw data, consuming unnecessary processing cycles and network bandwidth.

Third, many known systems are not configured to treat the interaction between a user and generated advice as structured feedback that is re-introduced into the computational pipeline. In such systems, user responses, such as ignoring, dismissing, or following recommendations, are either not captured at all, or are stored only as unstructured logs that are not used to update prompt construction logic or the behavior of analytical models. This lack of closed-loop adaptation prevents the system from improving its performance over time at the computer-system level, and results in repetitive or irrelevant advice that diminishes user engagement.

Fourth, when generative AI models are used, prior systems often submit simplistic prompts that omit important context such as long-term lifestyle trends, health history, or relationship patterns. This causes the generative model to produce generic, non-personalized output. From a technical perspective, this is an inefficient exploitation of a computational resource, because the model is not guided by a structured, machine-generated description of the user's state. Furthermore, the absence of a systematic scheme to include past analysis results and historical data in the prompt makes it difficult to control and reproduce AI outputs, hindering traceability and maintainability of the system.

Accordingly, there is a need for an improved computer-implemented system that (i) unifies acquisition and time-series storage of heterogeneous individual information, (ii) performs machine-driven aggregation, cleaning, and pattern analysis of the data prior to generative processing, (iii) constructs structured prompt sentences incorporating both raw profile information and machine-learning analysis results as an intermediate computational representation, and (iv) captures and feeds back user operation information regarding generated advice to automatically update at least one of the prompt generation logic and the analytical models. Such a system would improve the computational efficiency, the stability, and the adaptability of the advice generation pipeline itself, and thereby improve the functioning of the underlying computer system rather than merely automating a mental process.

The specific processing by the specific processing unit 290 of the data processing device 12 in Example 1 is realized by the following means.

The present invention provides a server comprising a processor and a storage device, the processor being configured to acquire heterogeneous individual information of a user from an information processing terminal and a measurement device, to store the individual information as time-series data in an information set in the storage device, to execute aggregation processing and preprocessing on the stored individual information by using a data processing program so as to generate statistical values, derived indexes, and trend information, to apply a machine-learning algorithm to at least the statistical values, the derived indexes, and the trend information to calculate prediction information or classification information as analysis information and store the analysis information in association with the information set, to construct an analysis input text including the analysis information and at least part of the individual information and generate a structured prompt sentence based on the analysis input text for input to a generative AI model, to input the prompt sentence to the generative AI model and acquire advice information in a natural language format from the generative AI model, to transmit the advice information as notification information to the information processing terminal, to acquire operation information of the user relating to the advice information from the information processing terminal and store the operation information as feedback information in the information set, and to update at least one of a rule for generating the prompt sentence and a parameter or configuration of the machine-learning algorithm on the basis of the feedback information so as to continuously improve generation accuracy and relevance of the advice information produced by the generative AI model. This enables an integrated computer-implemented pipeline in which heterogeneous sensor and behavioral data are automatically normalized and analyzed prior to generative processing, in which structured prompt sentences incorporating machine-derived analysis information are programmatically constructed and supplied to a generative AI model, and in which user interaction with generated advice is captured as feedback that reconfigures both prompt generation and analytical modeling, thereby improving the technical performance, efficiency, and adaptability of the underlying health-advice generation system.

The term “processor” refers to a hardware computation unit, such as a central processing unit or an execution core, that is configured to execute computer-readable instructions to implement the functions described in the present specification.

The term “storage device” refers to a non-transitory computer-readable medium, such as a semiconductor memory, a magnetic storage device, or an optical storage device, that is configured to store data and programs used by the processor.

The term “information processing terminal” refers to an electronic device having a processor and a display device, such as a mobile communication device, a portable computing device, or a stationary computing device, that is configured to communicate with the server and present information to a user.

The term “measurement device” refers to an electronic apparatus equipped with at least one sensor, such as a physiological sensor, a motion sensor, or an environmental sensor, that is configured to measure physical quantities relating to a user and to output corresponding measurement data.

The term “individual information” refers to user-specific data including at least lifestyle information, health status information, preference information, interest information, and information relating to human relationships of the user.

The term “lifestyle” refers to patterns of daily behavior of a user, including but not limited to sleep patterns, activity levels, nutritional habits, work schedules, and leisure activities.

The term “health status” refers to information indicative of a user's physical or mental condition, including but not limited to vital signs, clinical measurements, self-reported symptoms, and health risk indicators.

The term “preferences” refers to information representing choices or tendencies of a user toward particular activities, content types, or behavioral options in daily life.

The term “interests” refers to information indicating subject matters or domains that attract the attention or curiosity of a user, such as thematic categories of activities, knowledge fields, or recreational domains.

The term “human relationships” refers to information describing the social connections of a user, including but not limited to family relationships, friendship relationships, and frequency or pattern of social interactions.

The term “time-series data” refers to a sequence of data items in which each data item is associated with a time stamp or time interval, and which represents temporal evolution of the corresponding information.

The term “information set” refers to a structured data collection stored in the storage device and associated with a particular user, the structured data collection including at least time-series individual information and analysis information relating to the user.

The term “data processing program” refers to a set of computer-executable instructions that, when executed by the processor, cause the processor to perform aggregation processing, preprocessing, and other transformation operations on data.

The term “aggregation processing” refers to an operation that combines multiple data items into summarized data, including but not limited to computing totals, averages, counts, or other summary statistics over a specified time period or data subset.

The term “preprocessing” refers to a set of operations that transform raw data into a form suitable for analysis, including but not limited to cleaning, normalization, outlier removal, interpolation, and feature extraction.

The term “statistical values” refers to numerical indicators derived from one or more data items by applying statistical operations, such as an average, a median, a variance, or a correlation value.

The term “derived indexes” refers to computed features obtained from one or more raw or aggregated data items, such as a composite score, a ratio, a trend indicator, or any other transformed measure that is not directly measured by a sensor.

The term “trend information” refers to data indicating temporal patterns or changes in values over time, such as increasing, decreasing, cyclic, or stable patterns computed from time-series data.

The term “machine-learning algorithm” refers to a computational procedure that uses training data to learn a mapping from input data to output labels, scores, or predictions, and that can be applied by the processor to new input data to generate prediction information or classification information.

The term “prediction information” refers to data representing an estimated future state or risk level of a user's health status or lifestyle, generated by applying a machine-learning algorithm to input data.

The term “classification information” refers to data indicating a category or class assigned to a user's state, behavior, or profile, generated by applying a machine-learning algorithm to input data.

The term “analysis information” refers to information representing a result of computational analysis performed on the individual information, including at least prediction information, classification information, statistical values, derived indexes, and trend information.

The term “analysis input text” refers to a machine-generated text representation that includes at least a portion of the individual information and the analysis information, and that is configured to serve as a basis for constructing a prompt sentence.

The term “prompt sentence” refers to a machine-generated text that is supplied as input data to a generative AI model in order to instruct the generative AI model to generate advice information according to specified conditions.

The term “generative AI model” refers to a trained computational model configured to generate natural language text or other content in response to input data including a prompt sentence.

The term “advice information” refers to natural language text generated by the generative AI model on the basis of a prompt sentence, the natural language text including at least one recommendation or suggestion relating to improvement of a user's health status, lifestyle, or human relationships.

The term “notification information” refers to data transmitted from the server to the information processing terminal to cause the information processing terminal to present, to a user, at least part of the advice information or an indication that advice information is available.

The term “display device” refers to a visual output unit of the information processing terminal, such as a liquid crystal display or an organic light-emitting display, that is configured to present information to a user.

The term “operation information” refers to data representing user interactions with advice information presented on the information processing terminal, including but not limited to selection, confirmation, completion, evaluation, or dismissal of the advice information.

The term “feedback information” refers to operation information stored in association with the information set and used to modify or update parameters or logic applied in subsequent data processing or advice generation.

The term “generation accuracy” refers to a measure of how relevant, consistent, and appropriate the advice information generated by the generative AI model is, in view of the individual information and analysis information of a user.

The term “rule for generating the prompt sentence” refers to configuration information, templates, or algorithms that specify how to construct a prompt sentence from at least the analysis information and the individual information.

The term “parameter or configuration of the machine-learning algorithm” refers to adjustable values or structural settings, such as model coefficients, thresholds, hyperparameters, or feature selection schemes, that determine how the machine-learning algorithm processes input data and produces output information.

In one embodiment, a server cooperates with an information processing terminal and one or more measurement devices to implement the claimed system. The server includes at least one processor and at least one non-transitory storage device. The terminal includes a processor, a memory, a communication interface, and a display device. The measurement devices include one or more sensor units such as heart rate sensors, motion sensors, and blood pressure sensors, and are configured to communicate with the terminal and/or the server.

The server uses commodity server hardware, such as a multi-core central processing unit, a main memory, and an optional graphics processing unit for acceleration of machine-learning inference. The server executes an operating system and middleware such as a web server framework and a relational database management system. For example, the server uses a relational database management system to store time-series individual information, analysis information, and feedback information in separate but related tables. The server uses a data processing framework implemented in a general-purpose programming language to execute aggregation and preprocessing operations on the stored data. The server also uses a machine-learning library to implement a classification model and a prediction model that operate on numerical features computed from the stored individual information. The server further uses an implementation of a generative AI model, such as a transformer-based neural network, to generate natural-language advice from a prompt sentence that encodes analysis information and selected parts of the individual information.

The terminal operates as an input and output interface for the user. The terminal executes a mobile or desktop application that displays input forms, graphs, and advice messages on the display device. The terminal uses its communication interface to send measurement data and manual input data to the server via a secure communication protocol, and to receive notification information and advice information from the server. The terminal further receives push notifications from a notification infrastructure and presents them on the display device. The terminal optionally caches received advice information and analysis summaries in a local storage unit to permit offline viewing.

The user operates the terminal to provide individual information. The user confirms access permissions for measurement devices, such as granting permission for the terminal to retrieve heart rate, step count, and blood pressure values. The user further inputs lifestyle information, such as sleep duration, exercise frequency, dietary information, and social activity frequency, by interacting with graphical user interface elements provided by the terminal. The user reads advice information presented on the terminal, and the user provides operation information such as marking advice as completed, helpful, or not helpful, or ignoring advice. The user may also provide textual comments on particular pieces of advice.

The server constructs an internal data model in which individual information is stored as time-series records associated with user identifiers. The server uses a database schema that includes an individual information table, a summary table, an analysis table, an advice table, and a feedback table. The individual information table stores raw records from the measurement devices and the terminal, each record including at least a timestamp, a parameter type (for example, blood pressure, heart rate, step count, sleep duration, or social interaction count), and a numerical or categorical value. The summary table stores aggregated values per time window, such as daily averages and weekly totals. The analysis table stores prediction information and classification information computed by the machine-learning algorithm, such as a hypertension risk score, an activity level class, and a social engagement index. The advice table stores natural-language advice entries generated by the generative AI model, together with references to the corresponding analysis information and prompt sentence. The feedback table stores user operation information, such as a rating of each advice entry and a completion flag.

The server uses a data processing program to perform aggregation and preprocessing on the stored individual information. The server transforms raw timestamped data into normalized features using a consistent time grid, for example by resampling step-count values into daily totals and computing moving averages of blood pressure over a seven-day window. The server handles missing values by using defined imputation rules, such as forward-filling within a limited time window or substituting median values per user over a past period. The server detects and removes outliers using statistical criteria, such as rejecting values that are more than a specified number of standard deviations away from a recent mean. The server then computes derived indexes, such as a sleep regularity index based on the variance of bedtimes, an activity intensity index based on gym visits and step counts, and a social interaction index based on the frequency of recorded meetings with family and friends.

The server applies a machine-learning algorithm to the derived features to obtain prediction information and classification information. In one embodiment, the server uses a gradient-boosted decision tree model or a logistic regression model implemented by the machine-learning library. The server uses features such as weekly average systolic blood pressure, weekly exercise minutes, sleep regularity, and social interaction index as input vectors. During model training, the server uses historical data labeled with outcomes such as “blood pressure improved,” “blood pressure worsened,” or “no significant change,” and the server minimizes a loss function such as cross-entropy loss or mean squared error by adjusting model parameters. The server uses gradient-based optimization to update model weights and uses regularization to prevent overfitting. The server may also perform model selection and hyperparameter tuning, for example by cross-validation over historical data.

The server stores the resulting model parameters in the storage device and uses them for inference. During inference, the server normalizes input features using scaling parameters determined during training, and the server computes a risk score or class label for each user. For example, the server computes a probability that the user's blood pressure will remain elevated, or assigns the user to a “moderate risk” or “low risk” class. The server further computes classification labels such as “highly active,” “moderately active,” or “sedentary” based on activity levels, and “socially connected” or “socially isolated” based on social interaction features.

The server constructs an analysis input text by combining the analysis information with selected individual information in a structured textual form. The server uses a template engine or string-composition logic to format the analysis input text into sections such as “Lifestyle,” “Health status,” “Interests,” and “Human relationships,” each section including one or more sentences that summarize numerical features and classification labels. For example, the server constructs a text containing sentences such as “The user sleeps about 8 hours per night, goes to the gym 3 times per week, and has a slightly high average blood pressure,” or “The user meets friends once a month and lives with family members.” The server then appends a directive part that specifies how the generative AI model should generate advice, such as the number of advice items and the desired style.

The server generates a prompt sentence by using the analysis input text as a base and appending instructions to guide the generative AI model. In one example, the server generates a prompt sentence such as:
“You are a health and lifestyle coach. Based on the following user profile, generate three specific and actionable pieces of advice to improve the user's health and quality of life.
Lifestyle: sleeps 8 hours per night, goes to the gym 3 times a week.
Health status: blood pressure is slightly high.
Hobbies: reading, watching movies.
Interests: health foods, fitness.
Human relationships: lives with family, meets friends once a month.
Provide advice in simple English, with 1-2 sentences per recommendation.”

In another example, the server generates a prompt sentence such as:
“Generate concrete, personalized advice to improve the user's health status. The user data includes lifestyle habits, current health conditions, hobbies, interests, and information about human relationships. Focus on practical and safe recommendations that the user can start within the next week.”

The server uses a generative AI model to transform the prompt sentence into advice information. In one embodiment, the server deploys a transformer-based neural network language model implemented in a deep-learning framework. The generative AI model includes an input embedding layer that converts tokens of the prompt sentence into vectors, a stack of self-attention and feed-forward layers that model dependencies between tokens, and an output layer that outputs probability distributions over possible next tokens. The server configures the model with parameters such as a number of layers, an attention dimension, a vocabulary size, and an activation function. The server executes the model in inference mode on a graphics processing unit or on a central processing unit, and the server controls generation by specifying a maximum token length, a temperature parameter, and a top-k or top-p sampling scheme.

The server applies a decoding algorithm to select token sequences according to the model's output probabilities and the configured parameters. The server may further constrain decoding by applying rules such as avoiding repetition beyond a threshold and limiting the output to a certain number of sentences. The server thus generates a natural-language advice text that includes recommendations structured as discrete sentences. For example, the model outputs sentences such as “To help lower your blood pressure, keep your current exercise routine but reduce salt in your meals by choosing low-sodium products,” or “Schedule a 30-minute walk on days you do not go to the gym, and consider meeting friends or family for a walk to increase both physical activity and social interaction.”

The server post-processes the generated advice text to ensure compliance with length and content constraints. The server trims extraneous phrases, removes undesired disclaimers, splits the text into separate advice items if needed, and attaches metadata such as creation time, related analysis information identifiers, and a model version label. The server stores the advice information in the advice table and generates notification information that references the stored advice entry. The server then transmits the notification information to the terminal, for example via a push notification infrastructure or a messaging protocol.

The terminal receives the notification information and requests the full advice information from the server. The terminal renders the advice information in a dedicated screen using graphical components. The terminal may highlight different topics, such as diet, exercise, or social relations, using color codes or icons. The terminal enables the user to mark each advice item as completed, to rate its usefulness, or to ask to see more details. The terminal sends the user's operation information to the server as structured feedback records that indicate the advice identifier, a rating value, a completion flag, and an optional comment.

The server records the feedback information in the feedback table and uses it to update at least one of the rule for generating the prompt sentence and the configuration of the machine-learning algorithm. For example, the server analyzes feedback to determine that shorter advice with concrete, time-bounded steps receives higher completion rates. The server then modifies the prompt sentence templates to explicitly request “short, time-bounded steps” and to limit the number of sentences per advice item. In another example, the server learns that users with certain activity patterns benefit more from advice focusing on sleep than on exercise, and the server adjusts feature weights or decision thresholds in the machine-learning algorithm to prioritize sleep-related features when selecting content for the analysis input text. The server may re-train the machine-learning algorithm periodically by incorporating feedback information as additional labels or weights in the loss function.

The server, by structuring the computational pipeline into distinct modules for aggregation, analysis, prompt construction, generative inference, and feedback-driven adaptation, improves the functioning of the computer system. The server reduces computational cost for the generative AI model by supplying pre-computed analysis information and a concise analysis input text, thereby decreasing the number of tokens that must be processed and the number of inference steps required. The server improves memory usage by storing time-series data and derived features in normalized database structures, which permits efficient retrieval of only relevant data for each generation cycle. The server improves communication efficiency by sending only aggregated summaries and advice identifiers between server and terminal, rather than streaming entire raw measurement histories.

The server further improves accuracy and stability of advice generation by using a machine-learning algorithm to pre-analyze patterns in the individual information, thereby providing the generative AI model with a filtered and structured representation of the user's state. This reduces the impact of noise and missing values on the generated advice, as compared to a system that directly feeds raw data into a generative model. The feedback loop, in which the server updates prompt-generation rules and model configurations based on user interaction data, allows the system to adapt automatically and improve over time without manual rule tuning by human experts. This adaptation occurs through concrete changes in parameters, templates, and feature weightings within the computational components of the server, rather than merely changing business rules.

The server, the terminal, and the measurement devices thus cooperate to realize a concrete technical implementation that goes beyond merely automating human judgment. The server uses specialized data structures, feature engineering, machine-learning models, and a transformer-based generative AI model, together with a feedback-driven configuration update mechanism, to enhance processing speed, advice relevance, and robustness. This architecture yields a computer-implemented system that is technically improved in terms of processing efficiency, memory management, communication load, and prediction accuracy, and provides a practical implementation for generating personalized health, lifestyle, and relationship advice based on heterogeneous time-series data.

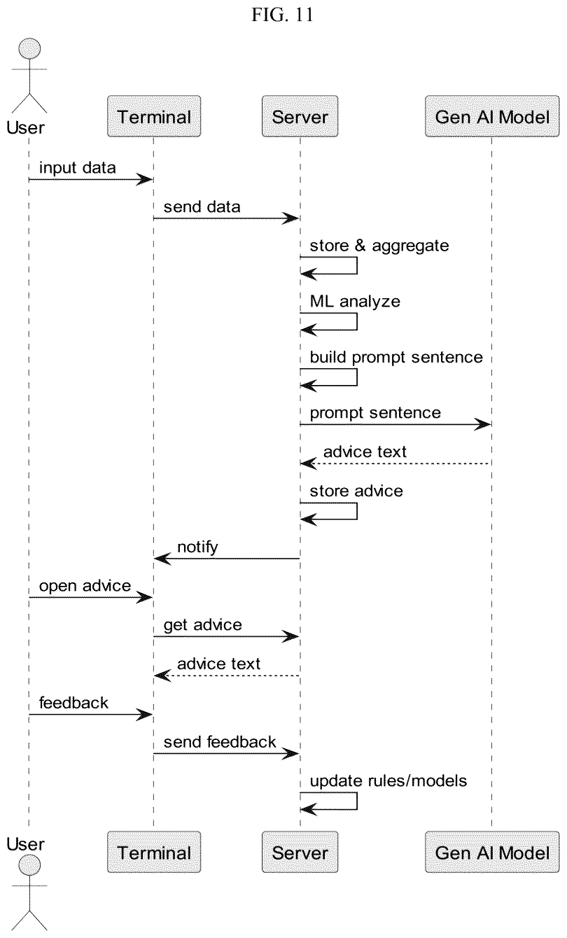

The following describes the processing flow using FIG. 11.

### Step 1:

The terminal acquires raw individual information from the user and from measurement devices and transmits the information to the server. The terminal receives, as input, sensor values such as heart rate, step count, and blood pressure from a measurement device, and manual entries such as sleep duration, gym frequency, and social activity logs from the user via a graphical user interface. The terminal packages these values into structured records including at least a user identifier, a timestamp, a parameter type, and a parameter value, and sends these records to the server via a secure communication protocol as the output of this step.

### Step 2:

The server receives and validates the raw individual information and stores the information as time-series data in a storage device. The server takes, as input, the structured records transmitted from the terminal, performs data validation such as type checking, range checking, and timestamp normalization, and rejects or corrects invalid entries. The server then writes the validated records into an individual information table in a relational database, assigning each record to a user identifier and storing it with a normalized timestamp as the output of this step.

### Step 3:

The server performs aggregation processing on the stored individual information to generate summary statistics for defined time windows. The server reads, as input, multiple time-series records from the individual information table for a given user and for a specified period, such as the last seven days. The server applies aggregation operations such as summing step counts per day, averaging systolic and diastolic blood pressure per day, and counting social interactions per week, and writes the resulting statistical values into a summary table indexed by user identifier and date as the output of this step.

### Step 4:

The server executes preprocessing and feature computation to generate derived indexes and trend information. The server uses, as input, the aggregated values from the summary table and the underlying time-series records from the individual information table. The server performs data processing such as computing moving averages, standard deviations, and coefficients of variation, and derives indexes such as a sleep regularity index, an activity intensity index, and a social interaction index by applying predefined formulas. The server further calculates trend indicators, for example, whether weekly exercise minutes are increasing or decreasing, and stores these derived indexes and trend information into an analysis feature table as the output of this step.

### Step 5:

The server applies a machine-learning algorithm to the derived features to obtain prediction information and classification information. The server takes, as input, feature vectors from the analysis feature table for each user, including statistical values, derived indexes, and trend indicators. The server normalizes these feature vectors using scaling parameters, feeds the normalized vectors into a trained classification or regression model such as a gradient-boosted tree or logistic regression model, and computes outputs such as a hypertension risk score, an activity level class, and a social engagement class. The server stores these outputs as analysis information, including prediction information and classification information, in an analysis table as the output of this step.

### Step 6:

The server constructs an analysis input text that summarizes the user's state based on the analysis information and the individual information. The server retrieves, as input, the analysis information from the analysis table and selected aggregated values and profile data from the summary table and individual information table. The server formats these elements into human-readable sentences grouped into sections such as lifestyle, health status, interests, and human relationships, by applying string concatenation and template filling operations. The server outputs a structured analysis input text that concisely describes the user's current condition and recent trends.

### Step 7:

The server generates a prompt sentence for a generative AI model by combining the analysis input text with generation instructions. The server uses, as input, the structured analysis input text from Step 6 and configuration information specifying the desired number of advice items, language style, and content focus. The server appends directive phrases to the analysis input text, such as requests to generate “three specific and actionable pieces of advice” or to focus on “practical and safe recommendations,” and produces a complete prompt sentence. The server stores this prompt sentence in association with the user and outputs it as the input data for the generative AI model.

### Step 8:

The server inputs the prompt sentence to the generative AI model and generates advice information in natural language. The server takes, as input, the prompt sentence produced in Step 7, tokenizes the text into tokens, and feeds the token sequence into a transformer-based neural network language model configured for text generation. The generative AI model performs internal computations using self-attention layers and feed-forward layers to compute probabilities for subsequent tokens, and the server applies a decoding algorithm such as top-k or nucleus sampling to select output tokens. The server concatenates the selected tokens to form advice text consisting of one or more recommendation sentences and outputs this advice text as advice information.

### Step 9:

The server post-processes and stores the generated advice information for later delivery and traceability. The server receives, as input, the raw advice text generated in Step 8. The server performs text processing such as trimming leading and trailing whitespace, segmenting the text into individual advice items if multiple recommendations are present, and enforcing length and formatting limits. The server then associates the advice information with the corresponding user identifier, analysis information identifiers, and prompt sentence identifier, and writes these records into an advice table in the storage device as the output of this step.

### Step 10:

The server prepares notification information and sends it to the terminal to inform the user of newly generated advice. The server uses, as input, the stored advice entries from the advice table and user notification preferences from a configuration table. The server constructs a notification payload containing at least an advice identifier, a short message such as “New health advice is available,” and timing information, and transmits this payload to the terminal via a notification infrastructure or a messaging protocol. The server outputs the notification payload to the terminal as the result of this step.

### Step 11:

The terminal receives the notification information and retrieves the corresponding advice information from the server. The terminal takes, as input, the notification payload including the advice identifier. The terminal initiates a network request to the server to fetch the full advice information associated with the advice identifier, receives the corresponding advice text and metadata in response, and stores or caches this data locally. The terminal outputs the advice information to its display logic for presentation.

### Step 12:

The terminal presents the advice information to the user and captures operation information representing user interaction. The terminal uses, as input, the advice text and metadata received from the server in Step 11. The terminal renders the advice text on the display device, organizes multiple advice items in a list or detail view, and provides user interface controls such as buttons or checkboxes for marking advice as completed, helpful, or not helpful. When the user operates these controls or closes the advice view, the terminal records the selected actions as operation information including at least the advice identifier, an action type, and a timestamp, and outputs this operation information to the server.

### Step 13:

The user interacts with the advice information and thereby generates feedback signals. The user receives, as input, the visual presentation of advice items on the terminal display. The user reads the advice, decides which recommendations to follow, and performs interface actions such as tapping “completed,” assigning a rating, or ignoring the notification. The user's actions result in operation information that the terminal encodes as feedback signals. The user thus indirectly outputs qualitative and quantitative feedback data that reflect perceived usefulness and applicability of the advice.

### Step 14:

The server receives operation information from the terminal and stores it as feedback information associated with the corresponding advice and user. The server takes, as input, structured feedback records including advice identifiers, action types, ratings, and timestamps. The server validates the records, assigns them to user identifiers, and writes them into a feedback table in the storage device. The server may also compute summary feedback metrics per advice item, such as total completions and average ratings, and store these as additional fields. The server outputs updated feedback data that can be used for subsequent adaptation.

### Step 15:

The server analyzes the feedback information to update prompt-generation rules and configuration parameters of the machine-learning algorithm. The server reads, as input, the feedback information from the feedback table, along with the corresponding advice content, prompt sentences, and analysis information. The server applies statistical and machine-learning techniques to detect relationships between prompt patterns, model settings, and user responses, such as identifying that shorter, more specific advice leads to higher completion rates. Based on these computations, the server modifies configuration data that control prompt templates, such as changing instructions in the prompt sentence to request a limited number of sentences, and adjusts parameters or feature weights in the machine-learning algorithm, such as increasing the importance of features associated with successful advice. The server writes the updated rules and parameters back to the storage device as the output of this step, thereby adapting future executions of Steps 5 through 8 to improve the relevance and accuracy of generated advice.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented health guidance systems typically apply fixed rule sets or simple statistical thresholds to sensor data and transaction records in order to generate health advice. In such systems, a processor usually evaluates biometric measurements and lifestyle logs in isolation, or with only coarse-grained aggregation of purchase data, and then selects pre-authored messages from a static template library. As a result, these systems suffer from several technical limitations.

First, conventional systems are not configured to transform heterogeneous, high-volume electronic transaction histories into structured behavioral feature quantities in a manner that is dynamically adaptable to a user's evolving habits. Raw transaction data, which may include merchant identifiers, product descriptors, timestamps, and amounts, is often stored as unstructured or weakly structured logs. Without systematic tabular conversion, category classification, and multi-dimensional aggregation, the processor cannot efficiently compute health-related indices such as category-wise purchase frequencies, time-zone-based behavioral patterns, or long-term transitions. This leads to suboptimal use of storage and processing resources, increased query complexity, and limited ability to derive meaningful behavioral features at scale.

Second, in many existing approaches, a generative AI model, if used at all, is invoked with generic or manually crafted prompts that do not embed machine-computed behavioral feature quantities or structured historical context. The prompt typically lacks explicit encoding of purchase frequency statistics by item type, temporal characteristics, and nutritionally oriented classifications. As a result, the downstream generative model cannot fully exploit the available data, and the quality, consistency, and personalization of the generated health advice are limited. From a computer-technology perspective, this represents an inefficient interface between the data processing pipeline and the generative model, because the model is not provided with an optimized, information-dense representation of the user's state.

Third, conventional systems do not tightly integrate the computation of structured behavioral feature quantities with prompt generation logic and notification control in a unified processing flow on a server. Instead, data aggregation, model invocation, and advice delivery are often implemented as loosely coupled modules or manual steps, leading to redundant data transformations, unnecessary input/output operations, and higher latency in generating and delivering advice. This fragmentation degrades system throughput, increases computation overhead, and impairs the scalability of the overall architecture.

Fourth, typical health advisory systems do not systematically produce machine-generated, user-specific action plans that are explicitly parameterized by computed behavioral feature quantities such as purchase counts per category, temporal purchase profiles, and classification results under nutritional viewpoints. Consequently, the output generated for different users or for different time periods is often generic, and the system architecture does not fully leverage the capability of generative models to synthesize advice contingent upon fine-grained, machine-derived features.

Accordingly, there is a need for an improved computer-implemented system and method in which a processor is configured to (i) acquire heterogeneous user-related data including biological information, lifestyle information, and electronic transaction history, (ii) transform the electronic transaction history into tabular data and compute behavioral feature quantities including health-related indices through classification and aggregation processing, (iii) generate structured, context-rich prompt sentences that encode analysis conditions and output formats for a generative AI model based on both individual information and behavioral feature quantities, and (iv) generate and deliver health support information specifying concrete changes in living behaviors, all in an integrated and automated pipeline. Such a system can improve the technical functioning of health guidance platforms by optimizing data representation, model invocation, and notification flows, thereby enabling more accurate, efficient, and scalable generation of personalized health advice.

The specific processing by the specific processing unit 290 of the data processing device 12 in Application Example 1 is realized by the following means.

The present invention provides a server comprising a processor and a storage device, the processor being configured to collect biological information and lifestyle information related to a user by using an information acquisition device and an input device, and to store the biological information and the lifestyle information as individual information in the form of structured information in the storage device; to acquire an electronic transaction history including commodity information and service information from a transaction information providing device or a terminal of the user, and to store the electronic transaction history as structured information in the storage device; to convert the electronic transaction history stored in the storage device into tabular data by using a data analysis program, and to perform classification processing and aggregation processing on the tabular data to calculate behavioral feature quantities including health-related indices; to generate a prompt sentence including analysis conditions and an output format relating to improvement of a health state and improvement of lifestyle habits on the basis of the individual information and the behavioral feature quantities, to input the prompt sentence into a generative information processing model, and to acquire a health-related analysis result from the generative information processing model; and to generate health support information that specifies contents of changes in living behaviors including eating behaviors and purchasing behaviors on the basis of the health-related analysis result, and to notify the health support information to the terminal of the user. This enables the server to technically improve processing of heterogeneous user data by converting electronic transaction histories into machine-computed behavioral feature quantities, to optimize interaction with a generative AI model through structured prompt sentences embedding such quantities, and to automatically produce and deliver user-specific, action-oriented health support information with reduced computational overhead, improved analysis accuracy, and enhanced scalability compared with conventional rule-based or manually configured health advisory systems.

The term “biological information” refers to information indicative of a physical or physiological state of a user, including, for example, body mass index, heart rate, blood pressure, sleep duration, activity level, or other measurement values obtained from a sensor device.

The term “lifestyle information” refers to information indicative of daily habits and preferences of a user, including, for example, exercise frequency, dietary habits, sleep patterns, work schedule, hobbies, and other routine behaviors that are not directly measured as biometric signals.

The term “information acquisition device” refers to a hardware or software component configured to obtain data relating to a user, including, for example, a sensor device, a wearable device, a communication interface, or a data input module that receives information from external services.

The term “input device” refers to a user interface component through which a user provides information, including, for example, a touch screen, a keyboard, a pointing device, a microphone, or a graphical user interface control that captures user entries.

The term “individual information” refers to a collection of data items that are associated with a particular user and that include at least biological information and lifestyle information, the collection being stored in a structured format in a storage device.

The term “storage device” refers to a computer-readable medium configured to store data and programs, including, for example, a magnetic storage medium, a semiconductor memory, a solid-state drive, or a database system implemented thereon.

The term “electronic transaction history” refers to a set of records representing transactions performed by a user through an electronic system, each record including at least information about a purchased commodity or a provided service, and optionally including a transaction date, a transaction amount, and an identifier of a transaction party.

The term “commodity information” refers to information indicating a purchased good in an electronic transaction history, including, for example, a product name, a product category, a quantity, or a unit price.

The term “service information” refers to information indicating a provided service in an electronic transaction history, including, for example, a service description, a service category, a time of provision, or a service fee.

The term “transaction information providing device” refers to a device or system that supplies electronic transaction history data to the server, including, for example, a payment processing system, a financial institution system, or an online commerce platform.

The term “terminal of the user” refers to a user-operated computing device configured to communicate with the server, including, for example, a smartphone, a tablet computer, a personal computer, or another network-enabled device.

The term “structured information” refers to data organized according to a predefined schema or format, such as records, fields, tables, or key-value pairs, that enables systematic querying, processing, and analysis by a computer program.

The term “data analysis program” refers to a software component executed by the processor and configured to transform and analyze data, including operations such as loading records, converting formats, classifying items, aggregating values, and computing indices.

The term “tabular data” refers to data arranged in a table structure composed of rows and columns, where each row represents an instance such as a transaction record and each column represents an attribute such as a date, a category, or an amount.

The term “classification processing” refers to processing in which items in tabular data are assigned to one or more categories or classes based on predetermined rules, patterns, or mappings.

The term “aggregation processing” refers to processing in which multiple data items are combined to produce summary values, including, for example, counts, sums, averages, or distributions aggregated over specified categories or time periods.

The term “behavioral feature quantities” refers to numerical or categorical values computed from user-related data that characterize behaviors of the user, including, for example, purchase frequencies, spending amounts, time-zone distributions, and other indices derived from electronic transaction history.

The term “health-related indices” refers to behavioral feature quantities that are interpreted in connection with a health state or risk of a user, including, for example, frequency of purchases of certain food categories, proportion of nutritionally beneficial items, or patterns of late-night transactions.

The term “prompt sentence” refers to a sequence of symbols, typically natural language text, constructed to specify analysis conditions, contextual information, and an output format, and provided as input to a generative information processing model to control its processing and output.

The term “analysis conditions” refers to parameters or instructions included in a prompt sentence that define how the generative information processing model should analyze input data, including, for example, a focus on particular behaviors, a target health goal, or a time range to be considered.

The term “output format” refers to a specification included in a prompt sentence that indicates a desired structure, style, or content organization of output generated by the generative information processing model, such as a list of action items, paragraphs of guidance, or bullet-point recommendations.

The term “generative information processing model” refers to a computation model implemented by software and executed on one or more processors, the model being configured to generate output information such as text based on input information including a prompt sentence and contextual data.

The term “health-related analysis result” refers to information output from the generative information processing model in response to a prompt sentence, the information including at least an interpretation of user behaviors and indications relating to a health state or lifestyle of the user.

The term “health support information” refers to information generated by the processor on the basis of a health-related analysis result, the information specifying one or more recommended changes or actions in the user's living behaviors to support improvement or maintenance of health.

The term “living behaviors” refers to behaviors performed by a user in daily life, including, for example, eating behaviors, purchasing behaviors, sleeping behaviors, and activity behaviors.

The term “eating behaviors” refers to behaviors of a user related to intake of food or drink, including, for example, choices of food categories, meal frequency, and timing of meals.

The term “purchasing behaviors” refers to behaviors of a user related to acquisition of goods or services, including, for example, categories of purchased items, purchase frequencies, transaction times, and spending patterns.

In one embodiment, a server implements the claimed system as a network-connected computing apparatus that cooperates with a terminal operated by a user. The server includes at least one processor, a memory, a storage device such as a relational database, and a communication interface. The terminal includes a processor, a memory, a display unit, an input unit such as a touch panel, and a wireless communication unit. The user interacts with the terminal to provide information and to view health support information generated by the server.

The server executes an application program that is implemented, for example, in a general-purpose programming language such as Python and that controls data acquisition, storage, analysis, prompt generation, interaction with a generative AI model, and delivery of health support information. The server uses a database engine such as a relational database management system to store structured information. In one example, the server uses a lightweight file-based database engine such as SQLite; in other embodiments, the server uses a client-server database engine. The server uses a data analysis library such as a table-oriented processing library (for example, a library corresponding to Pandas) to perform conversion of transaction logs into tabular data and to execute classification processing and aggregation processing.

The server acquires biological information and lifestyle information related to the user as individual information. The terminal presents graphical user interface screens that prompt the user to input lifestyle information such as daily meal patterns, exercise habits, and sleep schedules. The terminal also receives biological information from a sensor device, such as a wearable device that measures heart rate or step count. The terminal transmits the information to the server via a secure communication protocol such as HTTPS. The server receives the transmitted information and stores the information as structured records in the storage device. In one arrangement, the server stores individual information in a table with fields including: user identifier, age group, health goal category, average daily step count, average sleep duration, and self-reported dietary preference.

The server also acquires electronic transaction history associated with the user. The server receives transaction records from a transaction information providing device, such as an online payment platform or a financial institution server, via an application programming interface. Alternatively or additionally, the terminal uploads electronic receipts or transaction logs obtained from other applications. Each transaction record includes at least a transaction timestamp, an item descriptor, a merchant category code, and a transaction amount. The server stores the transaction records as structured information in one or more database tables. This structure allows the server to perform index-based searches and to execute join operations with individual information efficiently.

The server converts the electronic transaction history into tabular data suitable for algorithmic analysis. The server loads recent transaction records into memory and instantiates a table object in the data analysis library. The server normalizes date and time fields into a unified timestamp format, and derives a time-zone attribute such as morning, daytime, evening, or late night. The server applies category mapping rules to assign each transaction to a food or service category that is relevant to health analysis, such as fast food, fresh vegetables, sugary beverages, staple foods, and health-related services. These rules are implemented as mapping tables managed in the database or as deterministic functions in the analysis program.

The server performs classification processing and aggregation processing on the tabular data. The server groups transactions by user identifier, by category, and by time period such as a seven-day window, and computes behavioral feature quantities including: number of transactions in each category, total spending per category, and proportion of spending on categories labeled as nutritionally beneficial or nutritionally adverse. The server also computes health-related indices based on domain-specific rules. For example, the server calculates a fast food frequency index as the count of fast food purchases per week and a vegetable intake proxy index as the count of vegetable-related purchases per week. The server stores these behavioral feature quantities as structured records in a separate feature table, which allows incremental updates and avoids repeated scanning of raw transaction tables. This division of raw data and feature data reduces processing time for repeated analyses and decreases memory consumption.

The server generates a prompt sentence for a generative AI model based on the individual information and the behavioral feature quantities. The server retrieves health goals from the individual information, such as weight management or reduction of sugar intake, and combines them with recent behavioral feature quantities. The server constructs a natural-language prompt sentence that specifies: (i) the user's goals, (ii) summarized purchase behavior, (iii) nutritional classifications, and (iv) the desired output form.

For example, the server generates a prompt sentence such as:
“You are a digital health coach. Analyze the following purchase summary and generate specific, practical advice to improve the user's health. The user's main goals are: weight loss and reducing sugar intake. Purchase summary for the last 7 days: fast food purchases=4, fresh vegetable purchases=1, sugary drink purchases=5. Provide 3 concrete suggestions that the user can follow this week.”

In another example, the server generates a prompt sentence such as:
“Analyze the user's purchase history and generate advice about healthy lifestyle habits. Purchase history (last 30 days, in list form): [burger combo, 4 times; salad, 1 time; fried chicken set, 3 times; cola, 6 times]. Please describe specific, realistic improvements in diet and daily habits.”

The server incorporates analysis conditions into the prompt sentence, for instance by instructing the model to focus on reducing particular categories or to respect specific constraints such as a maximum budget or dietary restrictions. The server also specifies the desired output format, such as a numbered list of action items of a certain maximum length. By embedding numerical feature quantities and explicit instructions in this manner, the prompt sentence becomes a compact representation of the user's current state and analysis requirements. This representation is not a mere recitation of raw data but a machine-computed synthesis that reflects health-related patterns that are not readily apparent from unprocessed transaction logs.

The server inputs the prompt sentence into a generative information processing model. In one embodiment, the generative information processing model is a neural-network-based language model having an encoder-decoder architecture with multiple layers of self-attention units. The model has been trained on large-scale text corpora using a learning method such as stochastic gradient descent with backpropagation. During training, the model minimizes a loss function such as cross-entropy over token sequences, and internal parameters (weights and biases) are updated iteratively to reduce prediction error. The model uses token embeddings to map input tokens, including tokens corresponding to health-related terms and food categories, into a vector space where semantic relationships are captured. The model then applies attention mechanisms to condition generated tokens on both the prompt sentence and previously generated context.

The server invokes the generative information processing model through a model interface. In one specific implementation, the server uses an external model hosting service that executes the neural network on dedicated hardware, such as graphics processing units or tensor processing units. In another implementation, the server executes a locally hosted model using its own accelerators. The server transmits the prompt sentence and configuration parameters, including a maximum token count and a sampling temperature, to the model interface. The configuration parameters are chosen to balance diversity and stability of the generated content. For example, a lower temperature can reduce variance in output and improve reproducibility for similar inputs.

The server receives a health-related analysis result from the generative information processing model. This result comprises one or more paragraphs of text that evaluate the user's current purchasing behavior and provide recommendations for changing behaviors.

The server stores the analysis result in the storage device together with the associated behavioral feature quantities. The association allows later auditability and facilitates evaluation of the relationship between input features and generated advice.

The server generates health support information by further processing the health-related analysis result and the behavioral feature quantities. The server parses the generated text to identify explicit action phrases, and it correlates these actions with specific categories and time periods. For example, the server identifies phrases such as “replace at least two fast food meals this week with home-cooked meals including vegetables” and “choose water instead of sugary drinks three times this week.” The server encodes these actions in a structured form that includes: target category, recommended change in purchase count, applicable time period, and priority level. The server then produces a combined representation that includes both the natural-language advice and structured action parameters. This dual representation allows the system to track whether subsequent transaction data indicates compliance with recommendations.

The server transmits the health support information to the terminal. The terminal receives the health support information, stores it locally if needed, and displays the natural-language advice and suggested actions on the display unit. The terminal may present an interactive screen that lists each recommended change together with controls allowing the user to confirm or schedule implementation. For example, the terminal may display a list entry stating “This week, reduce fast food purchases from four times to two times,” and another list entry stating “Add at least two purchases of fresh vegetables.” The terminal may further present a progress indicator in later sessions based on updated behavioral feature quantities from the server.

The server's processing pipeline yields several technical effects. By converting dense transaction logs into normalized tabular data and precomputing behavioral feature quantities, the server reduces the computational cost of repeated analyses. Future analyses can be limited to newly added transaction records, and existing feature tables can be updated incrementally. This reduces latency in producing updated advice and allows the system to serve many users without proportional increases in processing time. In addition, the alignment between table structure and index structure in the database reduces input/output overhead and accelerates query execution.

By embedding behavioral feature quantities and explicit analysis conditions into the prompt sentence, the server provides the generative information processing model with a compressed, information-rich context that focuses the model on health-relevant aspects of user behavior. This improves the accuracy and consistency of generated advice as measured, for example, by alignment with domain rules and by reduction in contradictory recommendations. The server can evaluate generated advice against validation rules, and advice not meeting a threshold can be discarded or corrected by adjusting the prompt sentence. This feedback mechanism further enhances reliability.

From a computer-technology standpoint, the described combination of feature computation, prompt construction, and neural-model invocation constitutes more than an automation of human expert behavior. Human experts do not naturally compute and maintain hundreds of feature dimensions per user across long-term transaction histories. The server, by contrast, computes structured behavioral feature quantities in a high-dimensional space and uses these features as inputs to a neural network configured to model complex conditional distributions. This architecture enables the system to scale to large populations and to capture subtle behavioral patterns that would be infeasible for manual analysis.

The server also reduces communication load between components. Because prompt sentences are based on precomputed behavioral feature quantities rather than full raw transaction histories, the server can transmit compact strings to the generative information processing model instead of transmitting entire transaction datasets. This decreases network traffic and latency between the server and the model-execution environment. Furthermore, storing feature quantities and prompt-generation parameters enables re-use of analysis contexts without retransmitting or recomputing all underlying data.

In another embodiment, the server uses an alternative neural network architecture as the generative information processing model, such as a recurrent neural network with long short-term memory units, or a hybrid architecture that combines convolutional and attention layers. In such embodiments, the server still constructs prompt sentences embedding behavioral feature quantities and analysis conditions, and the network still uses trained weights updated via a sequence prediction loss. The choice of architecture can be adjusted based on computational resources and desired output characteristics.

In yet another embodiment, the server uses different feature sets or classification schemes. For example, the server may classify transactions according to sodium content, fat content, or fiber content inferred from a nutritional database. The server then computes health-related indices such as high-sodium-purchase frequency or low-fiber-purchase frequency. These expanded feature sets are incorporated into the prompt sentences in a similar fashion, allowing the generative information processing model to generate more specialized advice, such as advice oriented toward cardiovascular health or digestive health.

In a further embodiment, the server implements a rule-based pre-filter that detects extreme or potentially risky behavioral patterns, such as sudden spikes in late-night high-calorie purchases. If such patterns are detected, the server modifies the prompt sentence to instruct the generative information processing model to prioritize safety-oriented recommendations. This mechanism demonstrates that the server is not blindly passing data through the model, but rather conditionally controlling model behavior based on algorithmically computed criteria.

The described embodiments focus on internal technical processing, including data structures, analysis algorithms, and model interaction protocols, rather than on business workflows. The server controls computation of feature quantities, construction of prompt sentences, configuration of neural network parameters, and controlled generation and delivery of health support information. These aspects collectively improve the functioning of the computer system as a health guidance platform through increased processing efficiency, enhanced analysis accuracy, reduced communication overhead, and scalability to large datasets, while providing concrete, user-specific guidance that can be acted upon in the real world.

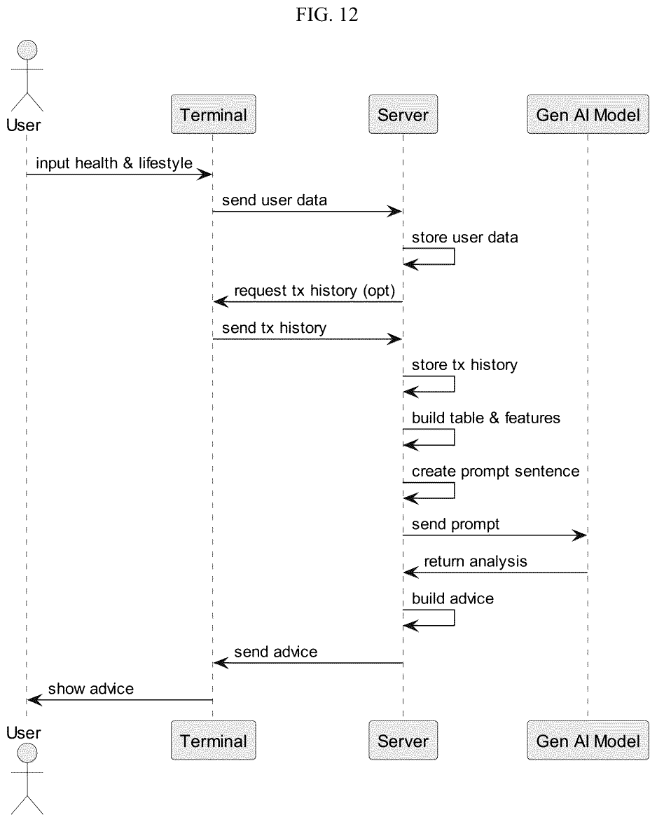

The following describes the processing flow using FIG. 12.

### Step 1:

The terminal collects individual information from the user and sends it to the server.
The user inputs lifestyle information such as meal frequency, exercise habits, and sleep duration via a graphical user interface on the terminal and optionally authorizes connection to sensor devices. The terminal receives biological information from a sensor device, such as heart rate and step count, through a wireless interface.
Input: user-entered lifestyle information and sensor-derived biological information.
The terminal packages these data items into a structured payload (for example, key-value pairs identifying age group, health goals, average daily steps, etc.) and transmits the payload to the server over a secure network connection.
Output: a structured data message containing individual information delivered to the server.

### Step 2:

The server stores the received individual information as structured records in a storage device.
   Input: structured individual information received from the terminal.
   The server validates field formats (for example, checking that numeric fields contain valid ranges, verifying that required health goal fields are present) and normalizes representations (such as converting time units and categorical labels into internal codes). The server then writes the normalized values into one or more database tables, associating them with a user identifier and indexing fields used for later queries.
   Output: structured individual information stored in database tables and indexed for retrieval.

### Step 3:

The server acquires electronic transaction history associated with the user.
Input: user identifier and connection information for a transaction information providing device, or transaction logs received from the terminal.
The server calls an external application programming interface or receives uploaded logs to obtain transaction records containing at least timestamps, item descriptors, merchant identifiers, and amounts. The server parses the records, converts date strings to timestamps, and maps external identifiers to internal formats. The server then stores the parsed records in transaction tables in the storage device, keyed by user identifier and transaction time.
Output: structured electronic transaction history stored in the database and linked to the user.

### Step 4:

The server converts the electronic transaction history into tabular data for analysis.
Input: transaction records retrieved from the database for a specified user and time range.
The server loads the records into memory and instantiates a table structure with one row per transaction and columns for attributes such as time, item description, merchant category, amount, and payment method. The server normalizes units, fills missing values where possible, and formats timestamps into a consistent internal representation.
Output: a tabular data structure representing the user's recent transaction history.

### Step 5:

The server performs classification processing on the tabular data to assign health-related categories.
Input: tabular transaction data with item descriptions and merchant identifiers.
The server applies rule-based mappings and lookup tables to classify each transaction into categories relevant to health analysis, such as fast food, fresh vegetables, sugary beverages, staple foods, or health services. The server may use string matching, category codes, and deterministic rules to select the appropriate category for each row. The server writes the resulting category labels into a category column in the tabular data.
Output: categorized tabular data in which each transaction is associated with a health-related category.

### Step 6:

The server performs aggregation processing to compute behavioral feature quantities and health-related indices.
Input: categorized tabular data for a target analysis period (for example, the last 7 or 30 days).
The server groups rows by category and by time window, counts the number of transactions per category, sums transaction amounts, and calculates ratios such as the proportion of spending on beneficial categories versus adverse categories. The server computes specific indices, such as weekly fast food purchase count, weekly vegetable-related purchase count, and frequency of late-night purchases. The server then stores these aggregated values as behavioral feature quantities in a feature table associated with the user.
Output: a set of behavioral feature quantities and health-related indices stored as structured data.

### Step 7:

The server retrieves individual information and behavioral feature quantities and constructs a prompt sentence for a generative AI model.
Input: user-specific individual information (health goals, preferences, constraints) and the behavioral feature quantities for a selected period.
The server formats the feature quantities into human-readable summaries and combines them with the user's goals. The server then generates a natural-language prompt sentence that explicitly states the user's goals, summarizes recent purchasing behavior, specifies which behaviors should be analyzed, and defines the expected structure of the output. For example, the server constructs a prompt sentence such as:
“You are a digital health coach. Analyze the following purchase summary and generate specific, practical advice to improve the user's health. The user's main goals are: weight loss and reducing sugar intake. Purchase summary for the last 7 days: fast food purchases=4, fresh vegetable purchases=1, sugary drink purchases=5. Provide 3 concrete suggestions that the user can follow this week.”
Output: a text-form prompt sentence containing analysis conditions, feature summaries, and output format instructions.

### Step 8:

The server transmits the prompt sentence to a generative AI model and obtains a health-related analysis result.
Input: the constructed prompt sentence and configuration parameters such as maximum output length and sampling temperature.
The server sends the prompt sentence to a neural-network-based generative information processing model through a model interface. The model consists, for example, of multiple layers of attention-based units trained via supervised learning to predict token sequences. The server provides the prompt as input tokens, and the model computes successive output tokens based on internal weights and the encoded context. The model's output is a sequence of text tokens that together form a health-related analysis result. The server receives this sequence, decodes it into text, and optionally checks for length, prohibited content, or format consistency.
Output: a health-related analysis result in natural-language text describing the user's current habits and recommended directions for improvement.

### Step 9:

The server derives structured health support information from the analysis result and the behavioral feature quantities.
Input: the health-related analysis result text and the previously computed behavioral feature quantities.
The server parses the analysis result to identify explicit recommendations, such as “reduce fast food meals by two per week” or “add two purchases of fresh vegetables this week.” The server maps these textual elements to structured fields including target category, recommended change in count, time horizon, and suggested priority. The server then constructs health support information that pairs the natural-language advice with these structured action parameters, ensuring that each recommendation is linked to corresponding behavioral feature quantities.
Output: health support information comprising human-readable advice and machine-readable action parameters.

### Step 10:

The server notifies the terminal of the generated health support information.
Input: health support information associated with the user identifier.
The server formats a notification payload that includes a title, a summary of the main recommendations, and identifiers supporting retrieval of full details. The server sends the payload to the terminal via a push notification service or a similar communication channel. The terminal receives the notification, stores any included details locally if necessary, and triggers the user interface to display a summary of the health support information.
Output: a notification delivered to the terminal that contains or references the health support information.

### Step 11:

The terminal presents the health support information to the user and allows user interaction.
Input: health support information received from the server.
The terminal displays the natural-language advice on a screen, showing each recommended action in a readable format and optionally rendering structured parameters such as target counts or time frames. The terminal may provide interactive elements that allow the user to acknowledge, save, or schedule specific recommendations. The user reads the presented advice and can indicate preferences or feedback through touch input.
Output: user-visible display of health support information and optional user feedback data.

### Step 12:

The server updates stored data and prepares for subsequent analyses based on new transaction history and user feedback.
Input: newly acquired transaction records, updated sensor data, and optional user feedback from the terminal.
The server appends new transaction records to the transaction tables, recomputes or incrementally updates behavioral feature quantities, and stores any feedback indicating whether previous recommendations were followed. The server may adapt prompt sentence generation rules based on observed responses, such as adjusting emphasis on particular categories or refining analysis conditions. These updated data and rules become the basis for future prompt sentences and analyses.
Output: refreshed individual information, transaction history, behavioral feature quantities, and configuration data stored in the database, enabling subsequent iterations of advice generation.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional health management systems typically collect sensor data from user devices and apply fixed, rule-based logic or static machine learning models to generate feedback. These systems suffer from several technical limitations in how information is processed and utilized by computers. First, the systems generally do not transform heterogeneous, time-series sensor data into a rich, structured context that can be effectively consumed by a generative AI model. As a result, the computing resources of the generative AI model are not efficiently leveraged, and the generated output is often generic and loosely related to the underlying data. Second, the systems lack a feedback loop in which user interaction data (such as viewing history, execution status, and user evaluation) is systematically captured and reintegrated into the computational pipeline. Without this loop, the computer cannot adapt its prompt formation or advice generation logic based on how users actually respond, leading to suboptimal use of storage, processing, and model capabilities over time. Third, prior systems do not provide a standardized, machine-implemented mechanism for dynamically constructing prompt sentences that encode numerical features, historical patterns, user attributes, and user reaction information in a form that conditions a generative AI model to perform domain-specific analysis and advice generation. This results in a technical inefficiency where the model must infer context implicitly from incomplete or unstructured prompts, increasing processing overhead and degrading output relevance.

There is therefore a need for improved computer-implemented techniques that (i) convert raw sensor data into structured feature sets and evaluation results; (ii) algorithmically construct prompt sentences that explicitly encode computed indices, user history, and behavioral reactions; and (iii) use these prompts to control a generative AI model in a manner that produces machine-processable advice objects. Such techniques should also provide a closed feedback loop in which user interaction data is stored and used to update the prompt construction logic and advice generation logic, thereby improving the efficiency, adaptability, and technical performance of the overall information processing system.

The specific processing by the specific processing unit 290 of the data processing device 12 in Example 2 is realized by the following means.

The present invention provides a server comprising a processor and a storage medium storing instructions, wherein the processor is configured to acquire, via a communication interface, time-series health-related information including physical activity information, physiological information, sleep information, and mental state information from an information acquisition device associated with a user; convert the acquired health-related information into structured data and store the structured data in a storage area of the storage medium; execute statistical processing and machine learning processing on the structured data to calculate feature values and evaluation results including activity indices, sleep indices, heart rate indices, and stress indices; construct a prompt sentence for input to a generative AI model based on the feature values and the evaluation results, the prompt sentence including information indicating a health state of the user, tendencies of lifestyle habits of the user, items requiring improvement, and output conditions, and transmit the prompt sentence to the generative AI model to obtain, from the generative AI model, an analysis result and candidate advice expressed in natural language; post-process the analysis result and the candidate advice to generate health advice information including summary information, a plurality of action proposals, category information, and priority information, and notify the health advice information to a terminal of the user; and acquire, from the terminal of the user, viewing history information, execution status information, and evaluation information related to the health advice information, store the viewing history information, the execution status information, and the evaluation information in the storage area, and update at least one of a configuration of the prompt sentence and contents of the health advice information based on the stored viewing history information, execution status information, and evaluation information. This enables the server to transform raw sensor streams into structured feature representations, to algorithmically generate and refine prompt sentences that condition a generative AI model in a controlled manner, and to form an adaptive feedback loop in which user interaction data is exploited to improve the efficiency, relevance, and technical performance of computer-implemented health advice generation.

The term “information acquisition device” refers to an electronic device including at least one sensor and a communication interface, which is worn or carried by a user and is configured to measure and output health-related information such as physical activity information, physiological information, sleep information, and mental state information as time-series data.

The term “health-related information” refers to data representing a physical or mental condition or lifestyle state of a user, including but not limited to physical activity information, physiological information, sleep information, and mental state information acquired from the information acquisition device.

The term “physical activity information” refers to data indicating movement or exercise performed by a user, including, for example, step counts, walking distance, energy expenditure, activity intensity, and body posture over time.

The term “physiological information” refers to biometric data representing a bodily state of a user, including, for example, heart rate, heart rate variability, respiration rate, body temperature, and related indices measured over time.

The term “sleep information” refers to data indicating a sleep state of a user, including, for example, sleep duration, sleep onset time, wake-up time, sleep stages, and sleep interruption events.

The term “mental state information” refers to data representing a psychological or stress-related condition of a user, including, for example, stress level, relaxation level, mood estimate, or related indices derived from sensor signals or user inputs.

The term “time-series data” refers to data composed of a sequence of values each associated with a time stamp, representing changes of health-related information of a user over time.

The term “communication path” refers to a logical or physical communication link, including wired or wireless networks, over which data is transmitted between the information acquisition device, a user terminal, and the server.

The term “information processing apparatus” refers to a computing apparatus including at least one processor, a memory, and a communication interface, which is configured to receive, store, and process health-related information.

The term “storage area” refers to a logical region of a memory or a storage medium in the information processing apparatus, which is allocated for storing structured data, health advice information, and user interaction information.

The term “structured data” refers to data arranged according to a predefined schema, such as tables, fields, and records, enabling programmatic access, querying, and processing by the information processing apparatus.

The term “statistical processing” refers to numerical computation applied to structured data, including, for example, aggregation, averaging, variance calculation, distribution analysis, and trend estimation of health-related information.

The term “machine learning processing” refers to executing an algorithm or model that has been trained on data, configured to infer feature values or evaluation results from structured data, including but not limited to classification, regression, or clustering models.

The term “feature value” refers to a numerical or categorical value derived from health-related information by statistical processing or machine learning processing, which is used as an input to further analysis or model evaluation.

The term “evaluation result” refers to an output of a computational analysis, derived from feature values, indicating an assessed state or score for a user, such as an activity level, a sleep quality level, or a stress level.

The term “activity index” refers to a feature value or evaluation result that quantitatively represents a degree of physical activity of a user over a specified period.

The term “sleep index” refers to a feature value or evaluation result that quantitatively represents a quality or quantity of sleep of a user over a specified period.

The term “heart rate index” refers to a feature value or evaluation result that quantitatively represents one or more characteristics of heart rate of a user, including, for example, average heart rate, resting heart rate, or heart rate variability.

The term “stress index” refers to a feature value or evaluation result that quantitatively represents a stress level or stress trend of a user over a specified period.

The term “prompt sentence” refers to a text sequence constructed by the system, including instructions, context information, and constraints, which is provided as an input to a generative AI model to control analysis and advice generation.

The term “generative AI model” refers to a machine learning model configured to generate natural-language text or similar content in response to an input prompt sentence, based on parameters learned from training data.

The term “health state” refers to a condition of a user's physical and mental state at a given time or over a period, derived from health-related information and expressed by indices, scores, or qualitative labels.

The term “lifestyle habits” refers to repetitive patterns of behavior of a user, including, for example, exercise habits, sleep schedules, work-rest balance, and stress management behaviors.

The term “items requiring improvement” refers to aspects of a user's health state or lifestyle habits that are determined, based on evaluation results or indices, to be suboptimal relative to predetermined criteria.

The term “output conditions” refers to constraints or requirements included in a prompt sentence that specify how a generative AI model should format, limit, or style its output, including, for example, output length, tone, or level of detail.

The term “analysis result” refers to a natural-language or structured output generated by the generative AI model in response to a prompt sentence, describing an interpretation or assessment of the user's health-related information.

The term “candidate advice” refers to one or more natural-language suggestions, proposals, or recommendations generated by the generative AI model, prior to post-processing by the system.

The term “post-process” refers to processing performed after receiving the analysis result and candidate advice from the generative AI model, including, for example, formatting, filtering, structuring, categorizing, or summarizing the generated content.

The term “health advice information” refers to data representing finalized advice generated by the system, including summary information, action proposals, category information, and priority information, which is intended to be presented to the user.

The term “summary information” refers to a concise textual or structured representation of a user's current health state or key findings derived from the analysis result.

The term “action proposal” refers to a recommended user behavior or step, expressed in natural language, that is intended to improve or maintain the user's health state or lifestyle habits.

The term “category information” refers to information indicating a classification of health advice information or action proposals into one or more categories, such as exercise, sleep, rest, or stress coping.

The term “priority information” refers to information indicating the relative importance or urgency of health advice information or action proposals, which can be used for ordering or emphasizing recommendations.

The term “terminal of the user” refers to an electronic device associated with a user, such as a mobile terminal, a portable terminal, or a computing terminal, which is configured to receive and display health advice information and to transmit user interaction information.

The term “viewing history information” refers to data indicating how and when the user has accessed or viewed health advice information on the terminal, including, for example, view timestamps and view durations.

The term “execution status information” refers to data indicating whether, how, or to what extent the user has attempted or completed one or more action proposals included in the health advice information.

The term “evaluation information” refers to data indicating explicit or implicit feedback from the user regarding the usefulness, relevance, or satisfaction level of the health advice information, including, for example, ratings or reaction selections.

The term “user interaction information” refers to information including at least one of viewing history information, execution status information, and evaluation information that is generated in response to the user's interaction with health advice information.

The term “history information” refers to information derived from past health-related information of a user over a predetermined period, including time-aggregated or trend-based representations of prior health states and behaviors.

The term “attribute information of the user” refers to information representing static or slowly changing properties of a user, including, for example, age group, biological sex, general activity level, or other demographic or profile data.

The term “reaction information” refers to a subset of user interaction information that indicates how the user responded to previously provided health advice information, including patterns in viewing, execution, and evaluation.

The term “reminder notification” refers to a machine-generated message delivered to the user terminal at a predetermined time or under predetermined conditions, prompting the user to perform or review an action proposal.

The term “achievement status” refers to information indicating whether a user has completed, partially completed, or not completed an action proposal within a specified period.

In one embodiment, the invention is implemented by cooperation of a server, a terminal, and a user.

The server includes at least one processor, a main memory, a non-volatile storage device, and a network interface. The server executes an operating system such as a general-purpose server operating system and middleware such as a web application framework and a database management system. The server further executes application software written, for example, in a general-purpose programming language and making use of a numerical computation library, a machine learning library, and a deep learning framework including a generative AI model.

The terminal includes a processor, a memory, a wireless communication interface, and a display unit. The terminal executes an operating system for mobile or portable devices, and executes an application program that communicates with the server, receives health advice information from the server, and displays the health advice information to the user.

The user wears or carries an information acquisition device, such as a wearable device or a mobile device, that includes one or more sensors such as an accelerometer, a gyroscope, a heart rate sensor, a temperature sensor, and a location sensor. The information acquisition device measures health-related information including physical activity information, physiological information, sleep information, and mental state information as time-series data. The information acquisition device stores the measured time-series data in a local storage area and transmits the data to the terminal or directly to the server through a wireless communication network.

The server receives the health-related information via the network interface. The server converts the received raw sensor data into structured data and stores the structured data in a relational database implemented by a database management system such as a relational database engine. In one example, the server defines tables for users, heart rate samples, step counts, sleep sessions, and stress scores. Each record includes a user identifier, a time stamp, a measurement value, and, optionally, a data quality flag.

The server uses a numerical computation library to load sets of records into memory as multi-dimensional arrays or table objects. The server applies statistical processing to compute aggregated measures. For example, the server computes, for each user, an average daily step count over a 7-day interval, a variance of sleep duration over a 14-day interval, an average resting heart rate during a specific time window, and a trend of stress scores using linear regression or moving averages. The server stores these computed values as feature values in a feature table in the database.

The server applies machine learning processing to the feature values. In one embodiment, the server uses a trained model implemented by a machine learning library such as a tree-based classifier or regressor to map the feature values to discrete labels such as “sedentary,” “moderately active,” or “active,” and to continuous scores such as a sleep quality score and a stress risk score. The server uses a feature vector that may include, for example, average steps for 7 and 30 days, standard deviation of bedtime, average sleep duration for 7 days, resting heart rate, and variance of stress indices by time of day. The server normalizes and scales each feature using predetermined scaling parameters, and passes the feature vector to the trained model to obtain an evaluation result for the user.

In another embodiment, the server uses a neural network model implemented by a deep learning framework. The neural network may be a feedforward network or a recurrent network with several hidden layers. Each hidden layer applies a non-linear activation function such as a rectified linear unit to provide non-linear mapping of inputs to outputs. The server uses a loss function such as mean squared error for regression of scores or cross-entropy for classification of lifestyle categories. During an offline training phase, the server updates model weights by a gradient-based optimization method such as stochastic gradient descent or an adaptive gradient method, using batches of historical user data. The server may also perform data augmentation by synthesizing additional training samples that simulate variations in activity patterns or noise in sensor readings, thereby improving model robustness and reducing overfitting.

The server uses the evaluation results and feature values to construct a prompt sentence to be input to a generative AI model. The generative AI model is, for example, an autoregressive language model or a transformer-based model trained to output natural-language text in response to a textual input. The server has stored parameters of the generative AI model in the storage device or has access to such a model via an external inference service.

The server constructs the prompt sentence according to predetermined rules. In one embodiment, the server concatenates sections including: (i) a role description of the generative AI model, (ii) a structured summary of the user's numerical indices and labels, (iii) a description of items requiring improvement, (iv) a list of applicable constraints, and (v) an explicit task instruction.

For example, the server constructs a prompt sentence such as:
“You are a health and lifestyle coaching assistant.
Here is a summary of one user's recent data:
Average daily steps in last 7 days: 3,800
Average sleep duration: 6.0 hours per night
Bedtime pattern: irregular (bedtime varies by more than 2 hours)
Resting heart rate: 78 bpm
Stress pattern: often high in the late evening
Overall activity level: sedentary
Sleep status: insufficient
Stress status: elevated
Task:
1. Briefly describe the user's current lifestyle in 2-3 sentences.
2. Provide 3-5 specific, practical recommendations to improve physical activity, sleep, and stress management.
3. Use simple, encouraging language.
4. Do not provide medical diagnoses or mention specific diseases.
5. Keep the total length under 250 words.”

In another example, the server constructs a daily advice prompt sentence such as:
“Use the following daily health metrics to generate concise advice for the user. Metrics:
Steps today: 3,200
Average steps in last 7 days: 3,800
Sleep last night: 5.5 hours
Average sleep in last 7 days: 6.0 hours
Resting heart rate: 80 bpm
Reported stress level: high in the afternoon
Task:
1. Briefly summarize the user's current status in 2-3 sentences.
2. Provide 3 specific recommendations (each 1-2 sentences) to improve physical activity, sleep, and stress.
3. Avoid medical diagnoses and use supportive, motivating language.”

The server transmits the constructed prompt sentence to the generative AI model via an internal interface or a network API. The server specifies decoding parameters such as maximum output length, sampling temperature, and penalty terms to control repetitiveness and diversity. The generative AI model, implemented as a multi-layer transformer network with attention mechanisms, processes the tokenized prompt sentence by repeatedly applying attention and feedforward transformations across layers to compute a probability distribution over possible next tokens. The server samples tokens according to the distribution or selects the most probable token at each step to generate the analysis result and candidate advice as text.

The server receives the generated text as an output sequence. The server then performs post-processing. For example, the server parses the generated text to identify a summary section, enumerated recommendations, and possible category endorsements such as “exercise,” “sleep,” or “stress management.” The server may apply rule-based parsing using regular expressions or natural-language parsing to segment sentences and detect line breaks or bullet markers. The server then structures the content into health advice information comprising fields for summary information, a list of action proposals, category information, and priority information. The server assigns priority values based on detected language patterns (for example, phrases such as “first,” “most important,” or “urgent”) and on internal rules mapping categories and risk levels to priority scores.

The server stores the health advice information in the database in a dedicated advice table. Each record includes a user identifier, advice text segments, categories, priority scores, creation time, and validity period. The server then notifies the terminal of the availability of new health advice information. The server uses a push notification service or a similar mechanism to send a compact notification payload containing a reference to the stored advice, such as an advice identifier and a short title.

The terminal receives the notification via a push messaging framework. The terminal authenticates with the server and requests the full health advice information using an identifier included in the notification. The terminal then receives the health advice information and stores it in local memory. The terminal displays the advice on the display unit in a user interface that shows the summary information and action proposals as selectable items. The terminal allows the user to set reminder notifications for specific action proposals and to enter execution status or evaluation ratings, such as marking an action as completed or indicating perceived usefulness.

The user interacts with the terminal by viewing the advice, setting reminders, and recording whether each recommendation is followed. In response, the terminal generates user interaction information including viewing history information, execution status information, and evaluation information, and transmits that information to the server.

The server receives the user interaction information and stores it in a user interaction table.
The server correlates each interaction record with one or more advice records and with the corresponding feature values and evaluation results that were used for generating the associated prompt sentence. The server then uses the stored user interaction information in subsequent prompt construction steps. For instance, the server may encode in a new prompt sentence how often the user has followed exercise recommendations compared to sleep recommendations, or may specify in the task description that the generative AI model should focus on categories with high acceptance by the user.

In one embodiment, the server constructs a prompt sentence including explicit references to user reaction patterns, such as:
“The user has consistently ignored previous recommendations about late-night screen time but has frequently followed walking recommendations.
When generating new advice, emphasize small, incremental changes to walking and light stretching, and de-emphasize recommendations about digital detox.”

By embedding such information into the prompt sentence, the server causes the generative AI model to condition its generation on user-specific interaction history. This construction of prompt sentences is performed systematically by the server based on stored, structured interaction data and pre-defined combination rules, which allows the computer system to adapt advice generation logic without retraining the generative AI model.

From a technical perspective, the server improves computer technology in several ways.

First, the server transforms heterogeneous raw sensor streams and interaction logs into structured feature representations and evaluation results using predetermined schemas and algorithms. This transformation enables efficient indexing, querying, and caching at the database layer, which reduces input size and complexity for the generative AI model and improves processing speed. Second, by separating numerical analysis (statistical and machine learning processing) from natural-language generation and by embedding analysis outputs into structured prompt sentences, the server reduces the need for the generative AI model to infer context from unstructured descriptions, thereby decreasing computational load inside the generative AI model and improving the reliability and precision of generated outputs.

Third, the server implements a closed feedback loop, in which user interaction information is fed back into the prompt construction logic and advice shaping logic at the server side. The server does not merely display static advice but updates internal control parameters that determine which features are highlighted in the prompt sentence, which categories are prioritized, and how strict certain constraints (such as output length or level of detail) are. This adaptive behavior is implemented by explicit rules and learned mappings in the server, and it leads to improved convergence speed and reduced iterations of advice generation because the generative AI model receives more precise constraints and context.

Fourth, in some embodiments, the server adjusts thresholds for feature selection based on interaction information. For example, if a particular feature such as bedtime variance shows little correlation with user response, the server can reduce its weight when constructing the prompt sentence, thereby limiting the inclusion of less relevant information. This selective inclusion reduces prompt length and token count, which in turn decreases inference time and communication bandwidth when the generative AI model is accessed via a remote service.

Fifth, the server implements non-conventional logic for prompt construction and post-processing. Instead of simply forwarding raw or minimally processed data to a generative AI model, the server generates a layered representation comprising: numeric indices; aggregated trends; discrete evaluation labels; user reaction statistics; and explicit task instructions. The server further parses the generative AI model output into a structured internal format that is reused across multiple modules, including scheduling modules for reminder notifications and modules for computing adherence statistics. This module structure enables file- and message-level reuse and reduces redundant computations, which contributes to improved overall system efficiency.

In a further embodiment, the server allows alternative models and algorithms. For example, the server may use a gradient boosting model instead of a neural network for evaluation results, or may use a different architecture for the generative AI model. The server may use a different loss function, such as a ranking loss, to train a model that orders candidate advice based on predicted adherence probability. The server may also implement different tokenization schemes or compression techniques for transmitting prompt sentences and generated outputs between the server and an external generative AI model service, thereby reducing communication load.

In another embodiment, the terminal performs some preprocessing. The terminal may pre-aggregate sensor readings into partial summaries and transmit only aggregated values, such as hourly step counts or nightly sleep blocks, to the server. In such a case, the server adjusts its statistical processing and feature extraction algorithms to operate on higher-level data, which can reduce network bandwidth and storage requirements at the cost of reduced temporal resolution. The system thus allows for configuration of aggregation levels to balance accuracy and resource usage.

In still another embodiment, the server executes an on-premise generative AI model instance instead of calling an external service. In that embodiment, the server loads model parameters from a local storage device into main memory and performs all tokenization, attention computation, and output generation locally. The server may adjust internal caching mechanisms for token embeddings and intermediate activations to reduce recomputation when generating similar prompts for multiple users, thereby further improving computational efficiency.

By implementing these structures and methods, the server, the terminal, and the user cooperate to realize a computer-implemented system that goes beyond simple automation of human decision-making. The system uses specific data structures, algorithms, and model configurations to transform raw sensor data and user interaction data into optimized prompt sentences and structured advice objects, and to control a generative AI model in a technically constrained and efficient manner. This results in improved processing speed, improved accuracy of advice relevance, reduced communication load, and better management of health-related information within the computer system.

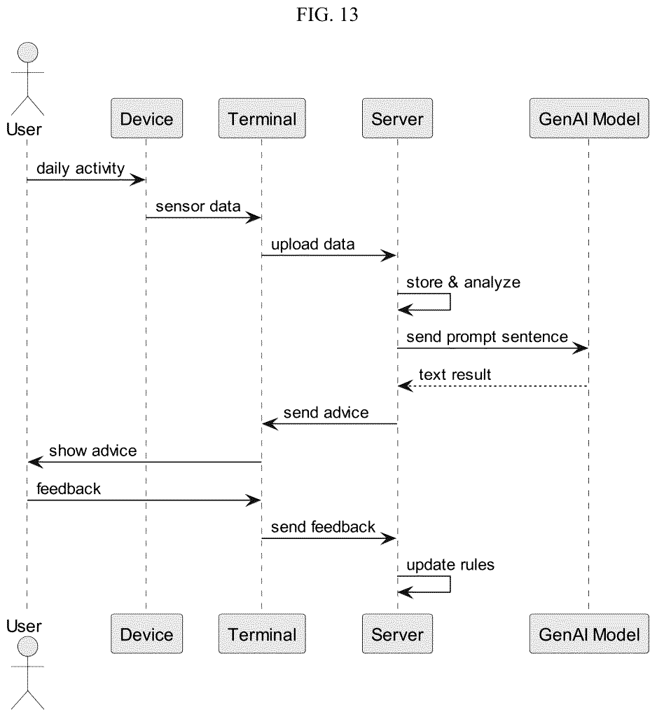

The following describes the processing flow using FIG. 13.

### Step 1:

The user wears or carries an information acquisition device and performs daily activities.
The input is the user's physical and mental state (for example, movement, heart activity, sleep, and stress).
The output is analog sensor signals within the information acquisition device, such as acceleration, optical pulse, body motion, and posture changes, which are sampled and digitized by the device hardware.

### Step 2:

The terminal acquires time-series health-related information from the information acquisition device.
The input is the digitized sensor signals provided by the information acquisition device through a short-range wireless connection or an internal API.
The terminal performs data acquisition and preprocessing, such as reading periodically sampled values, attaching timestamps from the terminal's clock, filtering out obvious noise, and converting raw sensor units into normalized units (for example, steps per interval, beats per minute, minutes of sleep).
The output is structured time-series records stored in the terminal's local memory, each record including at least a user identifier, a timestamp, a measurement type, and a measurement value.

### Step 3:

The terminal prepares upload data for transmission to the server.
The input is the locally stored time-series records that have not yet been uploaded, identified by an upload flag or a last-upload timestamp.
The terminal groups the records into a batch based on a time window or a maximum record count, compresses or aggregates values when applicable (for example, computing step totals per 5-minute window), and encodes the batch into a structured payload such as a text-based object including arrays of (timestamp, value) pairs for each measurement type.
The output is a formatted payload that includes aggregated and/or raw health-related information, a user identifier, and a time range, ready to be sent to the server.

### Step 4:

The terminal transmits the prepared payload to the server through a communication path.
The input is the formatted payload including batched health-related information and authentication data such as a token.
The terminal encrypts the payload using a secure protocol, encapsulates it in a network request, and sends it over a wireless or wired network to a predefined endpoint of the server.
The output is an encrypted network message delivered to the server's network interface.

### Step 5:

The server receives and authenticates the incoming request.
The input is the encrypted network message containing the payload and authentication data.
The server terminates the secure protocol, decrypts the message, validates the authentication data, and verifies that the user identifier in the payload matches a registered account. If validation succeeds, the server accepts the payload for further processing.
The output is a verified, decoded payload object in the server's memory, containing the health-related records associated with a particular user.

### Step 6:

The server converts the received health-related information into structured data and stores it in a database.
The input is the verified payload object containing arrays of time-stamped measurement values.
The server parses the arrays, maps each element to a database schema (for example, tables for heart rate, steps, sleep sessions, and stress indices), and performs data validation such as range checks and time consistency checks. The server then executes insert or update operations to store each record as a row, possibly aggregating fine-grained samples into fixed intervals for efficient querying.
The output is a set of structured database records, each record indexed by user identifier and timestamp, and marked as successfully stored.

### Step 7:

The server generates feature values and evaluation results from the stored structured data.
The input is a set of database records for a given user over a defined analysis period (for example, the last 7 days or 30 days).
The server executes statistical processing, such as calculating averages, medians, variances, minima, maxima, and trends. The server further executes machine learning processing, such as feeding a feature vector into a trained model to estimate scores and labels (for example, activity level, sleep quality, and stress risk). The server uses mathematical operations including vector multiplication, non-linear activation, and probability estimation, as defined by the selected model.
The output is a feature set and an evaluation set for the user, including numeric indices (for example, average daily steps, average sleep duration, resting heart rate, stress index) and categorical labels (for example, “sedentary,” “insufficient sleep,” “elevated stress”).

### Step 8:

The server identifies items requiring improvement and determines advice categories.
The input is the feature set and evaluation set output from the previous step, as well as predetermined threshold values or rules stored in the server.
The server compares each index with corresponding thresholds or reference ranges, detects indices that fall outside a preferred range, and maps these to improvement items such as exercise, sleep regularity, or stress management. The server also selects advice categories for each improvement item, for example mapping low step counts to “exercise” and short sleep duration to “sleep.”
The output is a list of identified improvement items and associated advice categories, each with an importance score based on deviation from the threshold and model-estimated risk.

### Step 9:

The server retrieves user history information, attribute information, and user interaction information.
The input is the user identifier and database tables that store prior health-related data, user profile attributes, and previous interaction logs (viewing history, execution status, and evaluation data).
The server performs database queries to obtain time-aggregated historical statistics, such as long-term activity trends, adherence rates to previous advice, and preferences inferred from past evaluations. The server also retrieves static or slowly changing attributes such as age group and general activity level.
The output is a history profile for the user that includes historical feature summaries, attribute values, and reaction patterns to previously delivered advice.

### Step 10:

The server constructs a prompt sentence for a generative AI model.
The input is the feature set and evaluation set for the current period, the list of improvement items and advice categories, and the history profile of the user.
The server generates textual segments describing (i) a role for the generative AI model, (ii) a numeric summary of the current indices, (iii) historical tendencies, (iv) specific improvement targets, and (v) explicit output constraints such as style, length, and prohibitions on medical diagnosis. The server then concatenates these segments according to predefined templates and rules, thereby transforming numeric and categorical data into a coherent text sequence.
The output is a fully constructed prompt sentence that encodes the user's health state, lifestyle habits, history, and desired form of output in a format suitable for input to the generative AI model.

### Step 11:

The server inputs the prompt sentence into the generative AI model and obtains an analysis result and candidate advice.
The input is the constructed prompt sentence and, optionally, decoding parameters such as maximum token count and sampling settings.
The server tokenizes the prompt sentence into discrete units, feeds these tokens into the generative AI model, and triggers inference. Inside the model, the server causes multiple layers of neural network computation to be executed, including attention weight calculation and non-linear transformation, to predict successive output tokens. The server then decodes the resulting token sequence back into natural-language text.
The output is a generated text that includes at least a natural-language analysis result describing the user's situation and candidate advice in the form of recommendations or suggested actions.

### Step 12:

The server post-processes the generated text to create structured health advice information.
The input is the natural-language text produced by the generative AI model.
The server parses the text to separate summary portions from recommendation portions, identifies sentence boundaries, and detects explicit or implicit ordering (such as “first,” “second,” “finally”). The server associates each recommendation with one or more categories (exercise, sleep, rest, stress coping, behavioral planning) based on keyword matching or pattern rules, and assigns a priority score using a combination of category importance and language markers.
The output is a structured health advice object containing summary information, a list of action proposals, associated category information, and priority information, suitable for direct storage and presentation.

### Step 13:

The server stores the health advice information and notifies the terminal.
The input is the structured health advice object and the user identifier.
The server writes the advice object into an advice table in the database, assigning a unique advice identifier and a creation timestamp. The server then constructs a notification message including at least the advice identifier and a brief title or preview text, and sends this message through a push notification service to the terminal linked to the user.
The output is a stored advice record in the server's database and a delivered notification message at the terminal.

### Step 14:

The terminal retrieves and displays the health advice information.
The input is the notification message that includes the advice identifier.
The terminal connects to the server using an authenticated request, fetches the full health advice information associated with the advice identifier, and stores it in local memory. The terminal renders the summary and action proposals on the display unit, for example as a list of items with category indicators and, optionally, buttons for setting reminders or marking completion.
The output is a visual presentation of the health advice information to the user and an internal representation of the displayed advice in the terminal's local storage.

### Step 15:

The user reviews the health advice and performs or rejects proposed actions.
The input is the displayed health advice information, including the summary and action proposals.
The user reads the content, decides whether to follow each action proposal, and may interact with the terminal by pressing buttons to set reminder notifications, to mark actions as done, or to provide evaluation feedback. The user's choices convert the human decision into discrete interaction events.
The output is human-initiated interaction signals captured by the terminal, representing user decisions and reactions to each recommendation.

### Step 16:

The terminal records user interaction information and transmits it to the server.
The input is the interaction signals generated by the user's operations on the terminal interface, including view events, completion marks, and evaluation scores.
The terminal encodes these events as user interaction records, each including at least a user identifier, an advice identifier, an interaction type, a timestamp, and any associated value (such as a rating). The terminal groups multiple interaction records into a batch and sends the batch securely to the server via the communication path.
The output is an interaction payload received by the server that describes how the user engaged with specific advice items.

### Step 17:

The server updates stored interaction data and adjusts future prompt construction and advice generation.
The input is the interaction payload containing batched user interaction records.
The server validates and stores each interaction record in a user interaction table. The server then aggregates interaction statistics for each user and each advice category, computing metrics such as adherence rate, average rating, and viewing frequency. Based on these metrics, the server modifies internal parameters and rules that control prompt sentence construction, such as weights for emphasizing certain categories, thresholds for including particular indices, and wording preferences for tone or detail level. These modifications alter how subsequent prompt sentences are formed and how generated texts are post-processed, thereby tailoring future advice to the user's demonstrated behavior.
The output is updated interaction statistics, updated configuration parameters for prompt sentence construction and advice shaping, and an improved basis for subsequent executions of Steps 7 through 12.

### Application Example 2

Description follows regarding a flow of the specific processing in an Application Example 2.

The units of the system described below are implemented by the data processing device 12 and the smart device 14. The data processing device 12 is called a “server” and the smart device 14 is called a “terminal”.

Conventional health-support systems that utilize user sensor data typically follow a fixed analytics pipeline implemented as static application logic. Such systems collect biometric signals from wearable devices, store those signals in a database, and apply predefined threshold rules or simple statistical models to derive recommendations. This architecture suffers from several technical limitations.

First, the recommendation generation logic is tightly coupled to the server application code. When additional data types such as behavioral information, emotional indicators, or purchase patterns are introduced, the analytics code and rule sets must be manually extended and redeployed. This creates a rigid system that scales poorly with growing data diversity and leads to frequent code changes and high maintenance costs.

Second, existing systems generally treat large-scale language models or other generative AI models, if used at all, as a peripheral component that simply turns precomputed results into fluent text. The prompt content is often static or handcrafted, and does not systematically encode a machine-readable representation of the user's historical health data, lifestyle patterns, and emotional state. As a result, the generative AI model cannot fully exploit the available context, which degrades the technical quality of its inference and forces the surrounding system to compensate with additional post-processing logic.

Third, there is no integrated mechanism for the system to use user feedback signals, such as interaction patterns or explicit ratings on received advice, to automatically refine the prompt construction logic and the underlying analysis models. Feedback is often stored in an ad-hoc manner or ignored, which prevents the system from iteratively improving the mapping from sensor data and historical records to prompt sentences and generated outputs. This leads to suboptimal personalization, inefficient use of computational resources, and repeated generation of low-relevance content.

Fourth, conventional database usage in these systems is largely passive: data is stored and queried, but not organized as unified “unique data” objects per user that combine biometric information, behavioral information, emotional state estimates, and purchase behavior over time. Consequently, the server must repeatedly perform expensive multi-table joins or cross-system queries to assemble context for each recommendation, which increases latency and reduces throughput under real-world load.

Therefore, there is a need for an improved computer-implemented system that (i) structures heterogeneous sensor and user-interaction data into unified unique data per user, (ii) analyzes this data using machine-learning techniques to produce compact, machine-readable analysis results, (iii) automatically constructs prompt sentences that encode these analysis results and historical context for a generative AI model, and (iv) uses user operation and evaluation feedback to dynamically refine the prompt construction and analysis models. Such a system would improve the technical functioning of the server by reducing coupling between analytics code and recommendation logic, improving the effectiveness and efficiency of generative AI-based inference, and enabling adaptive optimization of the overall processing pipeline.

The specific processing by the specific processing unit 290 of the data processing device 12 in Application Example 2 is realized by the following means.

The present invention provides a server comprising a processor and a storage apparatus, the processor being configured to acquire, from measuring apparatuses and input apparatuses, biometric information, behavioral information, and purchase information related to a user; to organize and store the acquired information in the storage apparatus as unique data structured per user and per time series; to extract feature values related to an activity amount, a heart rate, a sleep state, an emotional state, and an intake tendency of the user from the stored unique data; to analyze the extracted feature values by using a machine learning model so as to generate an analysis result that evaluates a health state, a lifestyle state, and the emotional state of the user; to construct, on the basis of the analysis result and the unique data, a prompt sentence that specifies analysis and advice-generation tasks for a generative AI model, the prompt sentence including summarized history information of the user and an estimated emotional state; to input the constructed prompt sentence to the generative AI model and acquire, as natural-language output, analysis result information and advice information relating to improvement of the health state, the lifestyle state, and a purchasing behavior of the user; to format the acquired advice information as notification message information, product proposal information, and service proposal information, and to transmit the formatted information to a terminal of the user via a communication apparatus so as to cause the terminal to display the advice information; and to acquire, from the terminal, operation information and evaluation information from the user with respect to the advice information, to store the operation information and the evaluation information in association with the unique data in the storage apparatus, and to update, on the basis of the stored operation information and evaluation information, at least one of a configuration rule for the prompt sentence and a parameter of the machine learning model used for the analysis. This enables the server to technically improve processing of heterogeneous user data by maintaining unified unique data objects, to generate context-rich and dynamically optimized prompt sentences for the generative AI model, to obtain higher-quality and more personalized advice with reduced application-level rule complexity, and to adapt the analysis and prompt-generation pipeline over time based on observed user interactions, thereby enhancing system performance, scalability, and the effectiveness of computer-implemented health and lifestyle support.

The term “processor” refers to a hardware or virtual computation unit, such as a central processing unit or an execution core in a computing apparatus, that is configured to execute instructions implementing the functions of acquiring, storing, analyzing, and transmitting data as described in the present specification.

The term “storage apparatus” refers to a hardware or virtual storage resource, such as a memory device or a persistent data store, that is configured to store user-related information, including unique data, operation information, and evaluation information, in a form accessible to the processor.

The term “measuring apparatus” refers to any sensing device, including but not limited to wearable devices, stationary sensors, and mobile device sensors, that is configured to measure biometric information, behavioral information, or environmental signals related to a user.

The term “input apparatus” refers to any user interface device, including but not limited to touchscreens, keyboards, microphones, and application interfaces, that is configured to receive explicit input from a user, such as text, selections, or feedback.

The term “biometric information” refers to data representing a physical or physiological state of a user, including, for example, heart rate, activity amount, sleep state, and other measurable bodily characteristics.

The term “behavioral information” refers to data representing actions, habits, or patterns of activity of a user, including, for example, movement patterns, daily routines, application usage patterns, and lifestyle-related behaviors.

The term “purchase information” refers to data representing transactions or purchasing behaviors of a user, including, for example, item identifiers, categories, purchase times, frequencies, and quantities of acquired goods or services.

The term “unique data” refers to a structured collection of user-specific information, including biometric information, behavioral information, purchase information, analysis results, and emotional state estimates, organized per user and in chronological order to represent an integrated profile of the user.

The term “feature values” refers to numerical or categorical representations derived from raw data within the unique data, such as averages, trends, counts, classifications, or other transformed indicators used as input to a machine learning model or analysis algorithm.

The term “activity amount” refers to a quantitative measure of a user's physical movement or exertion over time, including, for example, step counts, movement durations, or intensity levels derived from sensor measurements.

The term “heart rate” refers to a value or sequence of values indicating the number of heartbeats of a user per unit of time, such as beats per minute, as measured or estimated by a measuring apparatus.

The term “sleep state” refers to data describing the duration, timing, or quality of a user's sleep, including, for example, total sleep time, sleep stages, or sleep interruptions over a given period.

The term “emotional state” refers to a representation of a user's psychological or affective condition, such as stress, calmness, fatigue, or other labeled emotions, estimated from biometric information, behavioral information, or user input by an analysis model.

The term “intake tendency” refers to a pattern or propensity of a user's consumption behavior, including, for example, frequencies or types of food, drink, or other consumable items inferred from purchase information or related data.

The term “machine learning model” refers to a data-driven computational model, trained on example data, that is configured to perform tasks such as classification, regression, clustering, or pattern detection on feature values extracted from unique data.

The term “analysis result” refers to output information generated by the machine learning model or related algorithms, including evaluations or classifications of a user's health state, lifestyle state, or emotional state based on feature values.

The term “health state” refers to an assessment of a user's physical condition, including indicators such as fitness level, potential stress load, or risk-related trends, derived from biometric information and analysis results.

The term “lifestyle state” refers to an assessment of a user's habitual behaviors and daily routines, including, for example, exercise habits, sleep patterns, and dietary tendencies, derived from behavioral information and analysis results.

The term “generative AI model” refers to an artificial intelligence model, such as a generative language model, that is configured to produce natural-language or other content in response to a prompt sentence specifying an analysis or advice-generation task.

The term “prompt sentence” refers to a structured natural-language or machine-readable instruction sequence that encodes context, such as analysis results, history information, and emotional state, and specifies to the generative AI model the type and constraints of the output to be generated.

The term “analysis result information” refers to content in natural language generated by the generative AI model that explains or interprets a user's state, including, for example, summaries of detected patterns or conditions.

The term “advice information” refers to natural-language content generated or post-processed on the basis of the output of the generative AI model, including concrete proposals, recommendations, or guidance for improving a user's health state, lifestyle state, or purchasing behavior.

The term “notification message information” refers to advice information or related content that is formatted for delivery via a notification mechanism, such as a push notification, in-application message, or similar user interface presentation.

The term “product proposal information” refers to information identifying one or more goods or services recommended to a user, including, for example, product categories, item descriptors, or links, selected based on the user's unique data and advice information.

The term “service proposal information” refers to information identifying one or more services or programs, including, for example, training sessions or wellness services, proposed to a user based on the user's unique data and analysis results.

The term “terminal” refers to any user-operated computing device, such as a mobile device, wearable device, or general-purpose computer, that is configured to communicate with the server, receive advice information, and present information to the user.

The term “communication apparatus” refers to any hardware or software interface, including network interfaces and communication protocols, that is configured to transmit and receive data between the server and one or more terminals or measuring apparatuses.

The term “operation information” refers to data representing interactions of a user with advice information or user interfaces, including, for example, selections, clicks, confirmations, or navigation behaviors recorded by the terminal.

The term “evaluation information” refers to data indicating a user's assessment of advice information or proposals, including, for example, ratings, feedback comments, or indicators of usefulness or satisfaction.

The term “configuration rule for the prompt sentence” refers to a set of conditions, templates, or parameter values that define how analysis results, history information, and emotional state information are combined and expressed when constructing the prompt sentence for the generative AI model.

In one embodiment, a server cooperates with one or more terminals and measuring apparatuses to implement the claimed system. The server includes a processor, a storage apparatus, and a communication apparatus. The terminal includes a processor, a user interface, and a communication apparatus, and is communicatively coupled to various measuring apparatuses such as wearable sensors, smart phones, and in-store devices.

The server executes a program stored in the storage apparatus. The program is implemented, for example, as software modules written in a high-level programming language and executed by the processor of the server. The program configures the processor to perform acquisition, structuring, analysis, prompt construction, generative AI invocation, and feedback-driven updating of models and prompt rules as described below.

The terminal executes an application program that cooperates with the server. The terminal application controls measurement and input, presents advice information to a user, and collects operation information and evaluation information from the user.

### 1. Hardware and Software Configuration

The server uses at least the following hardware and software components:
(1) The server uses a computing platform including a multicore central processing unit, a main memory, and a network interface. In a practical implementation, the server may be realized by a cloud computing instance.
(2) The server uses a storage apparatus implemented as a database system and an object storage. In one example, the server uses a document-oriented database to store unique data per user, and a relational database to manage model configurations and prompt templates.
(3) The server uses a machine learning framework, such as a tensor computation library, to implement a machine learning model. The server implements a neural network model that receives feature values and outputs evaluation scores representing a health state, a lifestyle state, and an emotional state.
(4) The server uses a generative AI model, such as a transformer-based language model providing an API. The generative AI model receives a prompt sentence as text and outputs natural-language text.
(5) The server uses a communication stack including HTTPS and a push notification service for bidirectional communication with the terminal.

The terminal uses at least the following hardware and software components:
(1) The terminal uses a mobile processor, a display, and input interfaces such as a touch panel and a microphone.
(2) The terminal uses sensors integrated in a smart device or connected via short-range wireless communication. The sensors may include an accelerometer, a gyroscope, an optical heart-rate sensor, and a sleep-tracking sensor.
(3) The terminal uses an operating system providing application programming interfaces to access sensor data and notification functions.
(4) The terminal runs an application that communicates with the server, displays advice information, and transmits user operation information and evaluation information to the server.

A measuring apparatus such as a wearable device or an in-store kiosk includes a sensor module and a communication module that can transmit measurements to the server or to the terminal.

### 2. Unique Data Structure and Data Processing

The server stores, in the storage apparatus, unique data for each user. The unique data is represented as a structured data object that includes, for each user and for each time unit, the following fields:
biometric information fields (for example, heart rate samples, step counts, sleep duration and sleep stages),
behavioral information fields (for example, activity labels, application usage categories, time-of-day patterns),
purchase information fields (for example, product category counts, timestamps of purchases, purchase price ranges),
analysis result fields (for example, activity level, stress score, lifestyle score),
emotional state fields (for example, emotion labels such as “stress” or “fatigue”, and confidence scores), and
feedback fields (for example, binary indicators of whether advice was followed, user ratings, and textual comments).

The server organizes this unique data per user and per time series. The server assigns a user identifier and a time index to each record. The server uses this data structure to efficiently access historical data in a sliding-window manner without performing expensive joins at query time. This improves data access latency and reduces main memory usage, because the server can retrieve and process only the minimal subset of unique data required for analysis.

The server implements data processing pipelines using the machine learning framework. The server converts raw measurements into feature values by applying time-series transformations. The server computes, for example, moving averages of step counts, variability measures of heart rate, distributions of sleep duration over recent days, and frequencies of purchasing items in specific categories during certain time slots. The server quantizes or normalizes these feature values to bounded ranges, which allows numerical stability during neural network inference.

The server further derives text-based features from user messages or comments. The server uses a text tokenization component that converts text into token sequences. The server then applies an embedding layer, for example a precomputed word embedding or a subword embedding, to obtain vector representations of words and phrases. The server uses these vector representations as input to an emotion-estimation sub-network.

### 3. Machine Learning Model Architecture and Training

The server implements the machine learning model as a multi-branch neural network. The server constructs one branch for time-series biometric features and another branch for text-based features. The server connects the branches to a shared fully connected layer that outputs evaluation values.

In one embodiment, the server uses a recurrent neural network or a temporal convolutional network as the biometric branch. The server inputs sequences of feature vectors representing recent days. The server applies convolutional or recurrent layers with activation functions such as rectified linear units to capture temporal patterns. The server outputs intermediate representations indicating trends such as decreasing activity or increasing heart-rate variability.

In another embodiment, the server uses a feed-forward network for aggregated biometric statistics when high temporal resolution is not required.

The server uses a text branch implemented, for example, as a small transformer encoder or a recurrent network that consumes embeddings of user messages. The server produces latent vectors that capture semantics related to stress expressions or fatigue statements.

The server concatenates outputs from the biometric and text branches, then passes the concatenated vector through fully connected layers. The server generates as outputs:
a health-state vector including scores such as “cardiovascular load” or “exercise deficiency”,
a lifestyle-state vector including scores such as “sleep regularity” or “sedentary time”, and
an emotional-state label distribution indicating probabilities for emotion classes such as “stress” or “calm”.

The server trains this network offline using historical data labeled with known states or proxy outcomes. The server minimizes a loss function that combines cross-entropy loss for emotion classification and mean squared error for continuous state scores. The server performs gradient-based optimization such as stochastic gradient descent with adaptive learning-rate adjustment. During training, the server applies regularization techniques such as dropout and batch normalization to improve generalization and reduce overfitting.

By implementing this specific neural network architecture and training procedure, the server improves the accuracy and robustness of state estimation compared to simple rule-based thresholds. The server can detect subtle combinations of features that correlate with user states, which is difficult to encode manually.

### 4. Generative AI Model and Prompt Sentence Construction

The server uses a generative AI model in the form of a transformer-based language model accessible via an application programming interface. The generative AI model receives a prompt sentence and returns generated text that includes analysis result information and advice information.

The server does not send raw sensor data directly as a prompt. Instead, the server first compresses the unique data and analysis results into a structured context string. This context string includes, for example:
summarized feature descriptions such as “average daily steps: 3,200 over last 14 days”,
evaluation labels such as “activity level: low” and “stress level: high”,
emotional state description such as “emotion: stress (confidence: 0.82)”, and
purchase pattern description such as “frequent late-night purchases of high-calorie snacks”.

The server constructs the prompt sentence by combining the context string and a task specification. The task specification describes what kind of advice or explanation the generative AI model should produce and imposes format constraints.

The server uses prompt templates stored in the storage apparatus. Each prompt template defines:
which analysis results and history summaries to include,
target advice domain (for example, exercise, sleep, or diet),
output length range, and
tone constraints (for example, “friendly, non-judgmental”).

The server fills the template with current values from the unique data and analysis results. For example, the server may construct the following prompt sentence:
“The user's average daily steps during the last 14 days are 3,200. The resting heart rate is 78 bpm and has been gradually increasing. The estimated emotion state is ‘stress’. As a health coach, generate three concrete, friendly exercise suggestions that are suitable for a beginner and can be done at home without special equipment. Limit the response to 3-4 sentences suitable for display as a mobile notification.”

In another scenario that focuses on diet and purchase history, the server may construct the following prompt sentence:
“Analyze the following behavior: the user has purchased high-calorie snacks such as chips and sugary drinks 12 times in the last 30 days, often after 9 pm. The user's activity level is low and the stress level is moderate. Generate advice that supports healthier eating habits and suggest specific healthier alternatives such as nuts, fruit, or low-sugar drinks. Present the advice in a friendly, non-judgmental tone suitable for a smartphone notification.”

By explicitly encoding pre-computed analysis results and constraints in the prompt sentence, the server reduces the computation burden on the generative AI model and ensures more deterministic, controllable outputs. The server thereby improves the effectiveness and consistency of generative model usage.

### 5. Feedback-driven Adaptation and Technical Effects

The server receives, from the terminal, operation information describing how the user interacted with the displayed advice information. For example, the terminal may send click events when the user opens a detailed view, dismisses a notification, or follows a link to purchase a product. The terminal may also send evaluation information such as star ratings or free-form comments.

The server stores this operation information and evaluation information in association with the unique data. The server aggregates these feedback signals over time and computes performance metrics per prompt template and per advice type, such as:
a click-through rate for each combination of context conditions and template,
a completion rate for recommended actions (for example, starting a suggested exercise), and
average ratings per advice category.

The server uses these metrics to adjust configuration rules for the prompt sentence and parameters of the machine learning model. For instance, the server may modify prompt templates to shorten messages when long messages repeatedly show low engagement. The server may also adjust thresholds used to decide when to trigger exercise-related prompts versus stress-relief prompts. The server may update model weights using incremental learning when consistent feedback indicates systematic errors, such as overestimation of stress under certain biometric conditions.

This feedback-driven loop yields a technical benefit: the server reduces unnecessary notifications, decreases network traffic, and improves the ratio of relevant messages. Because the server adapts both prompt construction rules and underlying model parameters based on numerical feedback signals, the system performs a form of self-optimization that goes beyond fixed rule-based behavior.

### 6. Interaction Between Server, Terminal, and Measuring Apparatus

The terminal operates as a data collection and presentation device. The terminal requests sensor data from measuring apparatuses through standardized interfaces. The terminal can combine, for example, accelerometer readings and optical heart-rate sensor data into local measurements. The terminal batches measurements and user input events and transmits them to the server through the communication apparatus.

The server performs heavy computations, including feature extraction, neural network inference, and generative AI interaction. By centralizing these computations, the server can take advantage of hardware acceleration and optimized libraries. The server also aggregates data from multiple sources such as wearable devices and in-store kiosks, enabling more comprehensive analysis than a single local device could perform.

When the server transmits advice information to the terminal, the terminal displays the information through the user interface. The terminal may, for example, show a notification containing a brief advice sentence and allow the user to tap to view details. In response to user operations, the terminal sends back events that the server interprets as operation information and evaluation information.

### 7. Technical Improvements and Non-conventional Use of Computing Resources

The server implements several technical improvements beyond simple automation of human tasks.

First, by structuring data as unique data objects and precomputing feature values and analysis results, the server reduces runtime query complexity and computation time when constructing prompts. This improves throughput and latency, especially under high user load.

Second, by designing specific neural network architectures and training procedures tailored to the combination of biometric and textual data, the server improves estimation accuracy compared to manual rules or generic models. This leads to more precise evaluation of health state, lifestyle state, and emotional state, which is critical for efficient prompt construction.

Third, by dynamically generating and updating prompt sentences based on feedback, the server improves the efficiency of generative AI usage. The server focuses generative computation on high-value situations and uses context-rich prompts that minimize trial-and-error generation. This reduces communication load with the generative AI service and lowers computational overhead.

Fourth, by collecting structured feedback and using it to update both prompt configuration rules and machine learning model parameters, the server realizes an adaptive pipeline that continuously improves technical performance. The system can reduce false positives in advice triggers, avoid redundant notifications, and increase adherence to recommended actions.

Fifth, the system uses AI and generative AI in a way that is not equivalent to simple human task automation. Human operators typically cannot manually integrate high-dimensional sensor data, rich history information, and nuanced emotional state labels into a consistent, machine-optimized prompt format. The server uses explicit data structures, numerical optimization, and algorithmic feedback loops to perform this integration at machine speed and scale. This results in improved computational efficiency and systematic behavior that humans cannot replicate consistently.

### 8. Variations and Alternative Embodiments

In another embodiment, the server can use different machine learning architectures. For example, the server may use a graph-based model when relationships among users or between users and locations are important. The server may also employ ensemble models that combine neural network outputs with gradient-boosted decision trees to improve robustness.

In another embodiment, the server can use different generative AI models or host such a model locally. The server may pre-fine-tune the generative AI model with domain-specific data to enhance quality for health and lifestyle contexts. The server may also restrict the generative AI model to specific decoding strategies, such as beam search with constrained vocabulary, to maintain safe and relevant outputs.

In another embodiment, the server can control physical devices based on advice information. For example, the server may adjust a smart lighting device to dim lights when sleep improvement advice is active, or may control a treadmill speed in a fitness facility according to an exercise plan derived from analysis results. In such configurations, the server transmits control signals to actuators, thereby directly affecting the physical environment and further increasing technical relevance.

In yet another embodiment, multiple servers may operate in a distributed manner. A front-end server may handle authentication and API requests, while a back-end analytics server performs neural network inference and generative AI interaction. A model-management server may manage versions of machine learning models and prompt templates. These servers may communicate over secure channels and share access to the storage apparatus, thereby achieving scalability and fault tolerance.

Through these embodiments, the server, the terminal, and the user cooperate to realize a system in which heterogeneous sensor data and user feedback are transformed into structured unique data, analyzed by specifically designed machine learning models, and converted into optimized prompt sentences for a generative AI model. The system thus achieves technical effects including improved analysis accuracy, reduced processing latency, efficient use of communication and computation resources, and adaptive optimization of the overall data-processing pipeline.

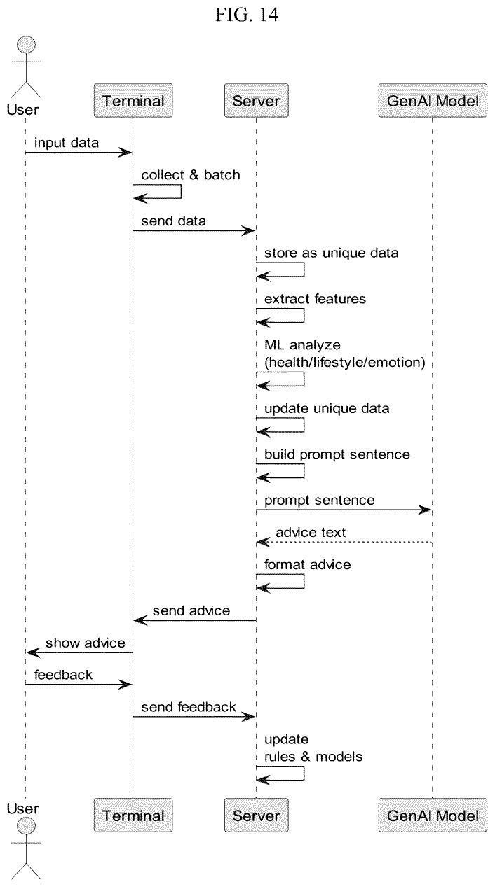

The following describes the processing flow using FIG. 14.

### Step 1:

The terminal acquires raw user data from measuring apparatuses and input apparatuses.
The terminal receives, as input, sensor readings such as heart rate samples, step counts, and sleep-tracking signals from wearable devices, as well as explicit user inputs such as text messages, questionnaire answers, and button selections from a user interface. The terminal performs data preprocessing operations including timestamping, unit normalization (for example, converting steps per interval to steps per day), and basic validation (for example, discarding clearly out-of-range values). The terminal then packs the preprocessed data into a structured record containing fields such as user identifier, time, biometric values, and text content. The output of Step 1 is a batch of structured data records buffered in the terminal.

### Step 2:

The terminal transmits the structured data to the server via a communication apparatus.
The terminal takes, as input, the batch of structured data records generated in Step 1 together with authentication tokens. The terminal opens an encrypted communication channel and performs a transmission operation in which it sends the batch as a serialized payload to a predefined server endpoint. During this operation, the terminal may compress the payload and attach metadata such as device type and firmware version. The output of Step 2 is a set of request messages delivered to the server that contain the user data batches ready for server-side processing.

### Step 3:

The server ingests the received data and stores it as unique data in the storage apparatus.
The server accepts, as input, the request messages received from the terminal in Step 2. The server first executes verification operations, including checking authentication tokens, validating user identifiers, and confirming schema conformity of the payload. The server then performs data transformation operations that map incoming fields to an internal unique data schema with explicit user and time axes. The server converts raw values into standardized representations (for example, computing daily aggregates from multiple intraday samples) and attaches internal keys for indexing. The server writes the transformed records into the storage apparatus as unique data organized per user and time series. The output of Step 3 is an updated unique data store in which the newly received records are integrated with existing historical records.

### Step 4:

The server extracts feature values from the unique data for analysis.
The server reads, as input, a subset of the unique data corresponding to a target user and a recent time window (for example, the last 14 days). The server performs numerical operations such as computing moving averages of step counts, standard deviations and variability indices of heart rate, histograms of sleep duration, and frequencies of purchases per item category and time-of-day segment. The server also generates categorical indicators such as “late-night snack present” or “workday exercise present” based on thresholding and rule evaluation. For text fields, the server tokenizes message content and converts tokens to embedding vectors using a pre-defined embedding table. The output of Step 4 is a feature set consisting of numeric feature vectors and text feature sequences suitable for input to a machine learning model.

### Step 5:

The server evaluates the user's health state, lifestyle state, and emotional state using a machine learning model.
The server takes, as input, the feature set produced in Step 4. The server executes a neural network model that includes, for example, a temporal branch for time-series biometric features and a text branch for encoded user messages. The server performs matrix multiplications, non-linear activations, and other layer operations defined by the model architecture. The server then computes output values such as continuous scores (for example, activity level score, sleep quality score) and probability distributions over emotion labels (for example, “stress”, “calm”, “fatigue”). The server may also compute a lifestyle pattern classification (for example, “sedentary” or “active”). The output of Step 5 is an analysis result containing evaluated health state, lifestyle state, and emotional state for the user.

### Step 6:

The server updates the unique data with the analysis result and emotional state.
The server receives, as input, the analysis result generated in Step 5 and the corresponding portion of unique data for the user. The server executes a merge operation that appends new fields such as health-state vectors, lifestyle-state vectors, and emotional-state labels to the time-indexed records. The server overwrites obsolete values, if any, and maintains version information for auditability. The server writes the updated records back into the storage apparatus. The output of Step 6 is an enhanced unique data profile in which raw measurements and derived analysis results are co-stored for subsequent use.

### Step 7:

The server constructs a context string and selects a prompt template.
The server uses, as input, the enhanced unique data profile from Step 6, including recent scores, labels, and summaries. The server performs a summarization operation that converts numerical analysis values into human-readable phrases such as “average daily steps: 3,200 over the last 14 days” or “stress level: high”. The server then selects a prompt template from a template store based on decision logic that inspects the analysis result (for example, choosing an exercise-oriented template when activity level is low, or a diet-oriented template when late-night snack frequency is high). The output of Step 7 is a pair consisting of a context string describing the user's current state and a selected prompt template defining the intended advice domain and format rules.

### Step 8:

The server generates a prompt sentence for a generative AI model.
The server receives, as input, the context string and the selected prompt template from Step 7.
The server performs a template-filling operation in which it substitutes placeholders in the template with concrete values extracted from the analysis result and unique data. The server may also append constraints regarding tone and length. For example, the server may generate a prompt sentence such as:
“The user's average daily steps during the last 14 days are 3,200. The resting heart rate is 78 bpm and has been gradually increasing. The estimated emotion state is ‘stress’. As a health coach, generate three concrete, friendly exercise suggestions that are suitable for a beginner and can be done at home without special equipment. Limit the response to 3-4 sentences suitable for display as a mobile notification.”
The output of Step 8 is a finalized prompt sentence text ready to be sent to the generative AI model.

### Step 9:

The server sends the prompt sentence to the generative AI model and receives generated text.
The server takes, as input, the prompt sentence produced in Step 8. The server performs an API invocation operation by transmitting the prompt sentence and control parameters such as maximum token count and randomness controls to the generative AI model endpoint. The generative AI model processes the prompt sentence and returns generated natural-language text. The server receives the response and parses it into distinct elements such as main advice paragraphs, bullet-point suggestions, and optional explanations. The output of Step 9 is a structured representation of generated analysis result information and advice information derived from the generative AI model's text output.

### Step 10:

The server formats the generated advice into notification, product proposal, and service proposal information.
The server uses, as input, the structured advice information obtained in Step 9 and the user's unique data including constraints such as dietary restrictions or preferred activity types. The server performs content filtering and mapping operations to remove unsuitable suggestions and to align proposed actions with the user's preferences and available resources (for example, only recommending services available in the user's region). The server then constructs message payloads adapted to different purposes: brief notification messages, product proposal entries (including item categories and links), and service proposal entries (including service names and possible schedules). The output of Step 10 is a set of formatted advice payloads tagged with delivery channels and priority indicators.

### Step 11:

The server transmits the formatted advice payloads to the terminal.
The server receives, as input, the formatted advice payloads from Step 10 and routing information associated with the user's terminal. The server uses the communication apparatus to send the payloads over a push notification service or a similar channel. The server may batch multiple payloads or schedule transmissions according to configured rules to avoid overloading the user. The output of Step 11 is a sequence of messages delivered to the terminal that contain the advice content and metadata needed for display.

### Step 12:

The terminal displays the advice information to the user and records operation events.
The terminal takes, as input, the messages received from the server in Step 11. The terminal performs a presentation operation in which it renders notifications on the display and, when the user interacts with them, opens detailed views containing full advice text, product descriptions, or service descriptions. The terminal also executes event-logging operations that capture user actions such as opening, dismissing, saving, or following links from the advice messages. The output of Step 12 is both the user's visual perception of the advice information and a set of locally recorded operation events representing the user's interaction with the advice.

### Step 13:

The user provides explicit evaluation information regarding the displayed advice.
The user receives, as input, the advice information presented by the terminal in Step 12. The user may then perform actions such as assigning a rating, selecting “useful” or “not useful” options, or entering free-form comments via input controls provided by the terminal. From a system perspective, the user's operations generate evaluation data containing identifiers of the advice items, numerical scores, and textual feedback. The output of Step 13 is a set of evaluation data elements stored temporarily in the terminal's memory.

### Step 14:

The terminal transmits operation information and evaluation information to the server.
The terminal uses, as input, the operation events from Step 12 and the evaluation data from Step 13. The terminal packages these events into a feedback payload that includes user identifiers, advice identifiers, timestamps, and the recorded feedback values. The terminal sends this payload to the server via the communication apparatus using a secure channel. The output of Step 14 is a feedback message received by the server containing operation information and evaluation information associated with specific advice instances and contexts.

### Step 15:

The server updates the unique data and refines prompt configuration rules and model parameters based on feedback.
The server accepts, as input, the feedback message transmitted in Step 14 and the current unique data and configuration state. The server writes the new operation information and evaluation information into the storage apparatus in association with corresponding time indices and advice identifiers. The server then performs statistical computations that aggregate feedback over multiple users and time periods, computing metrics such as engagement rates and average ratings for each prompt template and advice type. Using these metrics, the server executes an optimization operation in which it adjusts parameters of the prompt configuration rules (for example, modifying which analysis conditions map to which templates, or changing typical message lengths) and, optionally, updates machine learning model parameters by performing incremental training steps when systematic biases are detected. The output of Step 15 is an updated configuration state and, when applied, updated model weights that influence future analysis, prompt sentence generation, and advice delivery in a technically improved and more efficient manner.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like.

The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

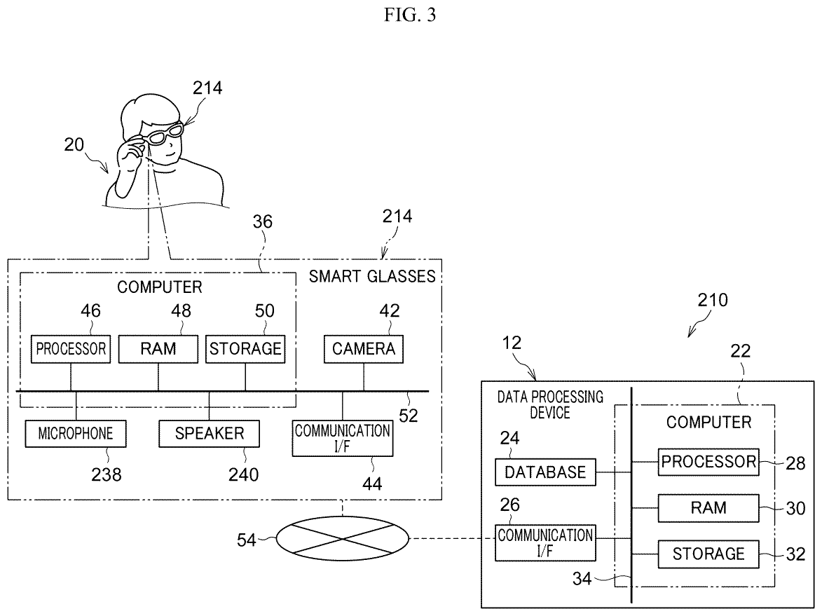

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

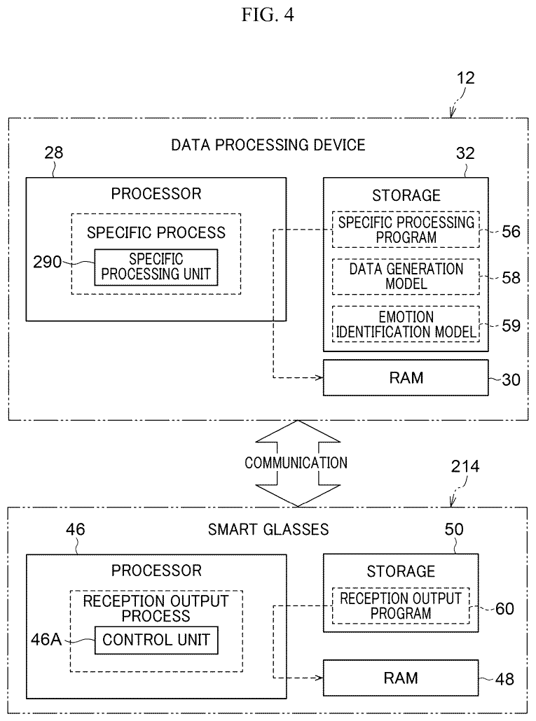

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like.

The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

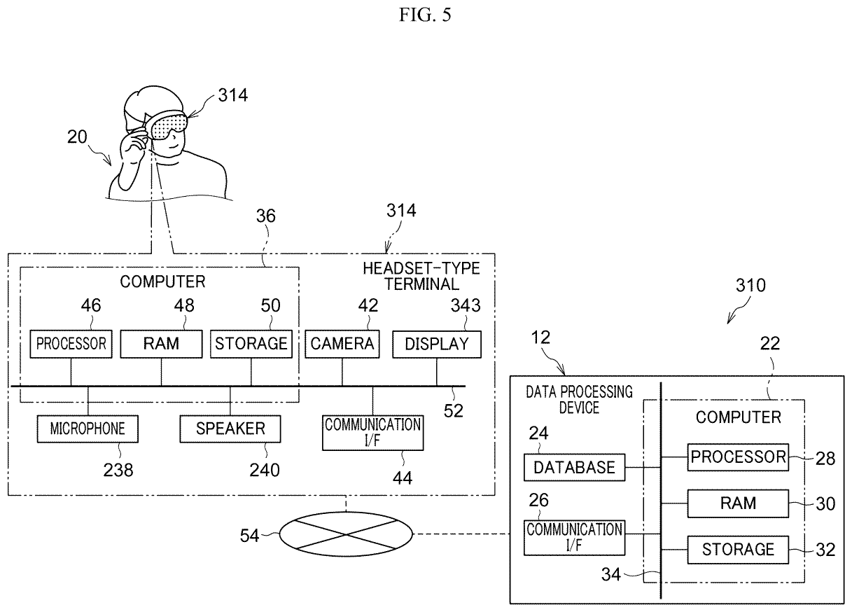

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

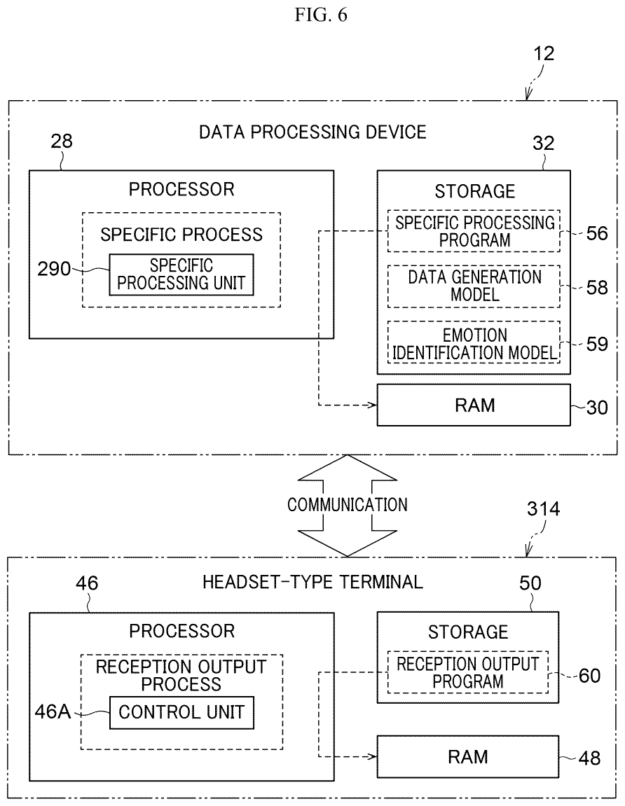

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like.

The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

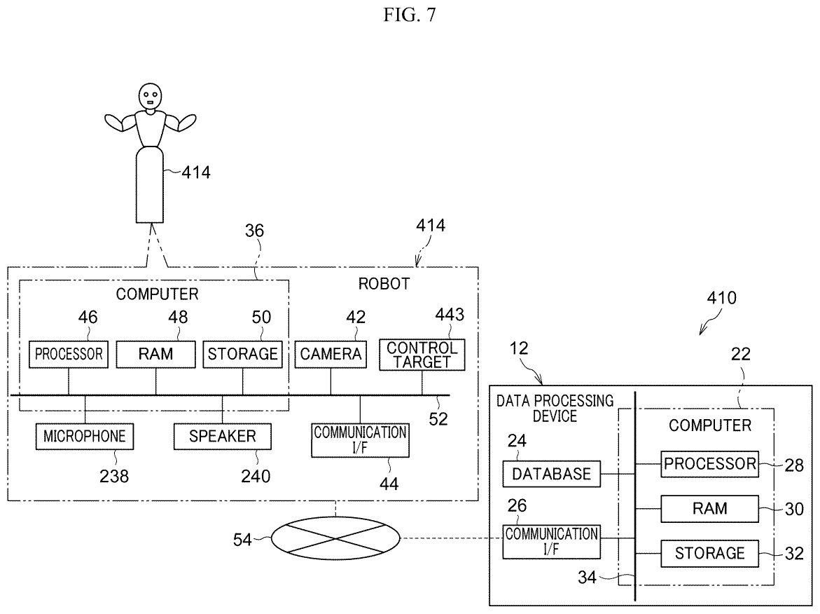

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

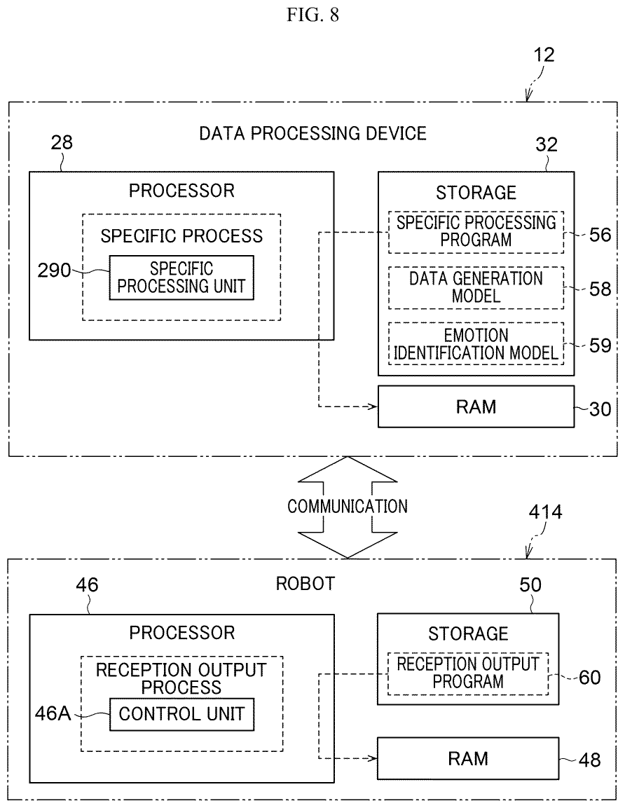

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like.

The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

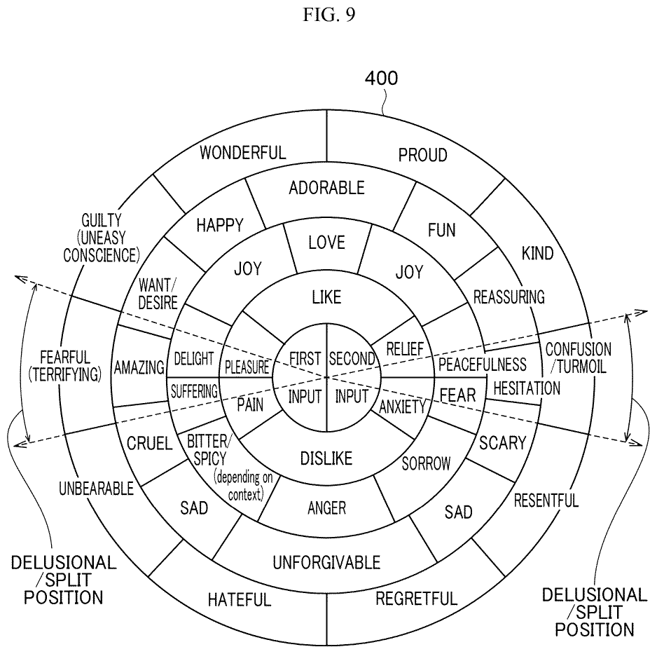

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

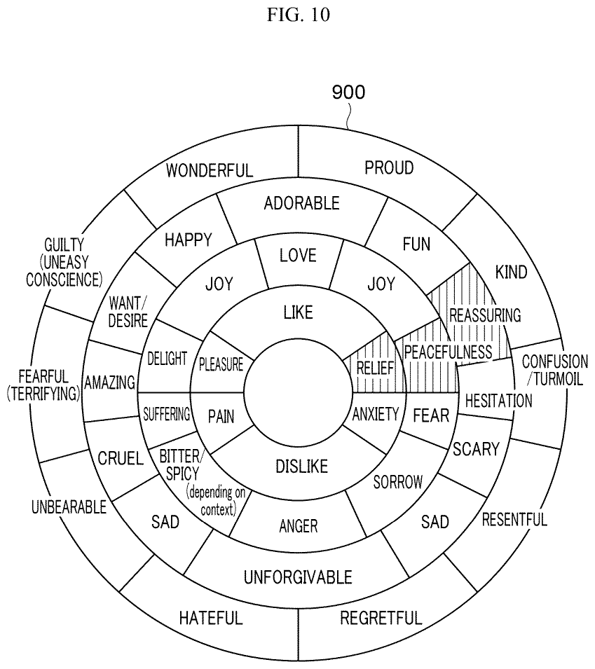

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
acquire individual information of a user from an information processing terminal and a measurement device, and store, in an information set in a storage device, time-series individual information including attribute information relating to lifestyle, health status, preferences, interests, and human relationships of the user, and
perform aggregation processing and preprocessing on the individual information stored in the information set by using a data processing program, and generate statistical values, derived indexes, and trend information, and store the statistical values, the derived indexes, and the trend information in association with the information set, and
calculate, on the basis of the statistical values, the derived indexes, and the trend information, prediction information or classification information relating to the health status and the lifestyle of the user by using a machine-learning algorithm, and store an analysis result as analysis information in association with the information set, and
construct an analysis input text including the analysis information and at least part of the individual information, and generate a prompt sentence to be used as input data to a generative AI model by using the analysis input text as a prompt sentence, and
input the prompt sentence to the generative AI model, and acquire, from the generative AI model, advice information in a natural language format relating to improvement of the health status, the lifestyle, and the human relationships of the user, and
transmit the advice information as notification information to the information processing terminal, and cause a display device of the information processing terminal to present the advice information, and
acquire operation information of the user relating to the advice information from the information processing terminal, and store the operation information as feedback information in the information set, and
update at least one of the prompt sentence and the machine-learning algorithm on the basis of the feedback information, so as to continuously improve generation accuracy of the advice information by the generative AI model.

### Supplementary 2

The system according to supplementary 1,
wherein the processor is configured to
generate the prompt sentence so as to instruct the generative AI model to perform analysis in consideration of history information of the health status and history information of the lifestyle stored as part of the individual information, thereby improving analysis accuracy of the generative AI model.

### Supplementary 3

The system according to supplementary 1,
wherein the processor is configured to
cause the advice information acquired from the generative AI model to include, on the basis of the analysis information, a plurality of concrete action proposals for improving the lifestyle and the human relationships of the user, and to generate the prompt sentence so as to cause each of the concrete action proposals to include executable action content and information relating to an execution period.

### Application Example 1

### Supplementary 1

A system comprising a processor,
wherein the processor is configured to
collect biological information and lifestyle information related to a user by using an information acquisition device and an input device, and store the biological information and the lifestyle information as individual information in the form of structured information in a storage device,
acquire an electronic transaction history including commodity information and service information from a transaction information providing device or a terminal of the user, and
store the electronic transaction history as structured information in the storage device, convert the electronic transaction history stored in the storage device into tabular data by using a data analysis program, and perform classification processing and aggregation processing on the tabular data to calculate behavioral feature quantities including health-related indices,
generate a prompt sentence including analysis conditions and an output format relating to improvement of a health state and improvement of lifestyle habits on the basis of the individual information and the behavioral feature quantities, input the prompt sentence into a generative information processing model, and acquire a health-related analysis result from the generative information processing model, and
generate health support information that specifies contents of changes in living behaviors including eating behaviors and purchasing behaviors on the basis of the health-related analysis result, and notify the health support information to the terminal of the user.

### Supplementary 2

The system according to supplementary 1,
wherein the processor is configured to
cause the prompt sentence to include purchase frequency information by item type extracted from the electronic transaction history, time-zone-based purchase information, and
classification information based on a nutritional viewpoint, and to include a description that instructs the generative information processing model, in the analysis, to take into account the individual information and a transition of past electronic transaction histories, thereby improving accuracy of the health-related analysis result.

### Supplementary 3

The system according to supplementary 1,
wherein the processor is configured to
cause the health support information to be generated, on a user basis, on the basis of the health-related analysis result acquired from the generative information processing model and the behavioral feature quantities, the health support information including concrete action proposals comprising increasing or decreasing, within a predetermined period, a number of purchases of a specific food category, switching a selection of a specific beverage category to an alternative, and changing a time zone of transactions.

### Example 2

### Supplementary 1

A system comprising a processor,
wherein the processor is configured to
acquire, by using an information acquisition device worn or carried by a user, health-related information including physical activity information, physiological information, sleep information, and mental state information as time-series data, and to store the acquired health-related information as structured data in a storage area of an information processing apparatus via a communication path, and
execute statistical processing and machine learning processing on the stored structured data to calculate feature values and evaluation results including activity indices, sleep indices, heart rate indices, and stress indices, and construct a prompt sentence for input to a generative AI model based on the feature values and the evaluation results, include in the prompt sentence information indicating a health state of the user, tendencies of lifestyle habits of the user, items requiring improvement, and output conditions, and transmit the prompt sentence to the generative AI model and obtain, from the generative AI model, an analysis result and candidate advice expressed in natural language, and
post-process the analysis result and the candidate advice obtained from the generative AI model to generate health advice information including summary information, a plurality of action proposals, category information, and priority information, and notify the health advice information to a terminal of the user, and
acquire, from the terminal of the user, viewing history information, execution status information, and evaluation information related to the health advice information, store the viewing history information, the execution status information, and the evaluation information in the storage area, and update at least one of a configuration of the prompt sentence and contents of the health advice information by using the stored viewing history information, execution status information, and evaluation information.

### Supplementary 2

The system according to supplementary 1,
wherein the processor is configured to
cause the prompt sentence to include, in addition to statistical values and feature values over a predetermined period extracted from the structured data, history information based on past health-related information, attribute information of the user, and reaction information of the user to the health advice information, and to include a description that instructs the generative AI model to perform analysis and advice generation in consideration of the history information and the reaction information.

### Supplementary 3

The system according to supplementary 1,
wherein the processor is configured to
cause the health advice information, based on the analysis result obtained from the generative AI model, to present, in natural language, a plurality of concrete action proposals related to exercise, sleep, rest, stress coping, and behavioral planning in association with execution frequency, execution time period, and execution conditions, and to provide the health advice information in a format that allows setting of reminder notifications and input of achievement status at the terminal of the user.

### Application Example 2

### Supplementary 1

A system comprising a processor,
wherein the processor is configured to
acquire biometric information, behavioral information, and purchase information related to a user by using a measuring apparatus and an input apparatus, and to store the acquired information in a storage apparatus as unique data organized for each user and in time series,
extract feature values related to an activity amount, a heart rate, a sleep state, an emotional state, and an intake tendency of the user from the stored unique data, and analyze the feature values by using a machine learning model so as to generate an analysis result that evaluates a health state, a lifestyle state, and the emotional state of the user,
construct a prompt sentence for causing a generative AI model to perform analysis and advice generation on the basis of the analysis result and the unique data, input the prompt sentence to the generative AI model, and acquire natural language analysis result information and advice information relating to improvement of the health state, the lifestyle state, and a purchasing behavior of the user,
format the acquired advice information as notification message information, product proposal information, and service proposal information, and transmit the formatted information to a terminal of the user via a communication apparatus so as to cause the terminal to display the advice information, and
acquire operation information and evaluation information from the user with respect to the advice information from the terminal, store the operation information and the evaluation information in the storage apparatus in association with the unique data, and update a configuration of the prompt sentence and a model used for the analysis on the basis of the stored operation information and the evaluation information.

### Supplementary 2

The system according to supplementary 1,
wherein the processor is configured to
include, in the prompt sentence, history information obtained by summarizing past biometric information and lifestyle information of the user included in the unique data, and the emotional state of the user estimated by the machine learning model, so as to improve analysis accuracy and individual optimization of the advice by the generative AI model.

### Supplementary 3

The system according to supplementary 1,
wherein the processor is configured to
include, in the advice information acquired from the generative AI model, alternative product information, behavior program information, and in-store service information selected on the basis of the unique data of the user in accordance with the health state and the lifestyle state, and to notify the user of concrete behavior proposals and product proposals relating to exercise, sleep, eating habits, and stress relief.

What is claimed is:

1. A system comprising:
circuitry configured to:
acquire, via a communication interface coupled to a packet-switched network, time-series data records from an information acquisition device associated with a user, each time-series data record including a timestamp and a measurement value;
convert the acquired time-series data records into structured data and store the structured data in a storage area;
execute statistical processing on the structured data to compute aggregated values over a plurality of time windows, and apply a trained inference model to at least the aggregated values to generate evaluation results representing assessed states of the user;
construct a prompt sentence for a generative language model based on the evaluation results and at least a portion of the structured data, the prompt sentence specifying output conditions constraining a format of a generated response;
input the prompt sentence to the generative language model and obtain, from the generative language model, a generated response expressed in natural language;
post-process the generated response to produce a structured output including a plurality of action proposals and associated priority information; and
transmit a notification data packet containing at least a portion of the structured output to a terminal device of the user via the communication interface.

2. The system according to claim 1, wherein the circuitry is configured to packetize the structured output into a plurality of data packets formatted according to a transport-layer protocol for transmission over the packet-switched network to a registered network address of the terminal device.

3. The system according to claim 1, wherein the circuitry is configured to acquire, from the terminal device via the communication interface, interaction data representing user operations performed in response to the structured output, store the interaction data in association with the structured data in the storage area, and update at least one of a configuration rule for the prompt sentence and a parameter of the trained inference model based on the stored interaction data.

4. The system according to claim 3, wherein the interaction data includes viewing history information indicating timestamps at which the user accessed the structured output, execution status information indicating whether the user performed at least one of the action proposals, and evaluation information indicating a rating assigned by the user to at least one of the action proposals.

5. The system according to claim 3, wherein the circuitry is configured to aggregate the interaction data over a plurality of feedback cycles to compute adherence metrics per action-proposal category, and adjust, based on the adherence metrics, at least one of a weighting applied to the evaluation results when constructing the prompt sentence and a threshold determining which evaluation results are included in the prompt sentence.

6. The system according to claim 1, wherein the circuitry is configured to compute, as part of the statistical processing, derived index values including a regularity index based on variance of timestamps within a subset of the time-series data records and an intensity index based on cumulative measurement values over a sliding time window.

7. The system according to claim 1, wherein the trained inference model comprises a multi-branch neural network including a temporal branch configured to receive sequential feature vectors derived from the time-series data records and a text branch configured to receive embedding vectors derived from textual input of the user, the temporal branch and the text branch connected to a shared layer that outputs the evaluation results.

8. The system according to claim 1, wherein the circuitry is configured to encode, in the prompt sentence, a role description for the generative language model, a numerical summary of the evaluation results, identifiers of items determined to require improvement based on comparison of the evaluation results with predetermined thresholds, and a constraint on a maximum token length of the generated response.

9. The system according to claim 1, wherein the post-processing includes parsing the generated response to identify discrete recommendation segments, associating each recommendation segment with a category label selected from a predefined set of category labels based on keyword matching, and assigning a priority score to each recommendation segment based on a deviation of a corresponding evaluation result from a reference value.

10. The system according to claim 1, wherein the circuitry is configured to acquire an electronic transaction history associated with the user from a transaction information source via the communication interface, convert the electronic transaction history into tabular data, perform classification processing and aggregation processing on the tabular data to compute behavioral feature quantities, and include the behavioral feature quantities in the prompt sentence.

11. The system according to claim 10, wherein the behavioral feature quantities include a purchase frequency per item category over a predetermined period, a time-of-day distribution of transactions, and a ratio of transactions classified under a first nutritional category to transactions classified under a second nutritional category.

12. The system according to claim 10, wherein the circuitry is configured to generate, based on the generated response and the behavioral feature quantities, formatted output including notification message information, product proposal information identifying recommended goods, and service proposal information identifying recommended services, and transmit the formatted output to the terminal device.

13. The system according to claim 1, wherein the circuitry is configured to apply an emotion identification model to at least one of audio data, text data, and physiological data associated with the user to estimate an emotional state of the user, and include the estimated emotional state in the prompt sentence.

14. The system according to claim 13, wherein the emotion identification model maps input data to emotion values according to an emotion map in which a plurality of emotions are arranged based on a structure giving rise to each emotion, and wherein emotions that co-occur are mapped to proximate positions in the emotion map.

15. The system according to claim 1, wherein the circuitry is configured to store the evaluation results in association with corresponding time-series data records in the storage area, and when constructing a subsequent prompt sentence, retrieve historical evaluation results over a predetermined past period and include trend information derived from the historical evaluation results in the subsequent prompt sentence.

16. The system according to claim 1, wherein the circuitry is configured to normalize the measurement values using scaling parameters determined during a training phase of the trained inference model prior to applying the trained inference model, the trained inference model trained by minimizing a loss function combining cross-entropy loss for categorical outputs and mean squared error for continuous score outputs.

17. The system according to claim 1, wherein the circuitry is configured to encode the notification data packet with a display controller command that causes the terminal device to render the plurality of action proposals as selectable interface elements, each selectable interface element associated with a control for setting a reminder notification and a control for recording an achievement status.

18. A system comprising:
circuitry configured to:
receive, via a communication interface coupled to a packet-switched network, time-series sensor data from a wearable device of a user, the time-series sensor data including physical activity data, physiological data, and sleep data;
store the time-series sensor data as structured records in a storage area, each structured record indexed by a user identifier and a timestamp;
compute feature values from the structured records by executing statistical processing including at least computing averages and variances over a sliding time window, and apply a trained classification model to the feature values to generate evaluation labels and continuous scores representing an activity level, a sleep quality, and a stress level of the user;
assemble a prompt sentence for a generative language model by concatenating a role description segment, a numerical summary segment encoding the evaluation labels and the continuous scores, an improvement-target segment identifying evaluation labels that fall outside predetermined reference ranges, and an output-constraint segment specifying a maximum response length and a prohibited-content directive;
transmit the prompt sentence to the generative language model via an application programming interface, receive a generated text from the generative language model, and parse the generated text into a summary portion and a plurality of recommendation portions;
assign to each recommendation portion a category label and a priority score based on the evaluation labels and keyword analysis of the recommendation portion;
packetize the summary portion and the plurality of recommendation portions into one or more transport-layer data packets and transmit the one or more transport-layer data packets to a registered network address of a terminal device of the user; and
receive, from the terminal device, feedback data packets containing interaction records that indicate at least one of a viewing event, an execution-status update, and a rating for at least one of the recommendation portions, and store the interaction records in the storage area in association with the structured records.

19. The system according to claim 18, wherein the circuitry is configured to aggregate the interaction records over a plurality of time periods to compute category-specific engagement metrics, and modify at least one of a template used for assembling the prompt sentence and a feature-selection rule applied prior to computing the feature values based on the category-specific engagement metrics.

20. A method performed by circuitry, the method comprising:
acquiring, via a communication interface coupled to a packet-switched network, time-series data records from an information acquisition device associated with a user;
converting the time-series data records into structured data and storing the structured data in a storage area;
executing statistical processing on the structured data to compute aggregated values, and applying a trained inference model to the aggregated values to generate evaluation results;
constructing a prompt sentence for a generative language model based on the evaluation results and at least a portion of the structured data; inputting the prompt sentence to the generative language model and obtaining a generated response;
post-processing the generated response to produce a structured output including a plurality of action proposals and associated priority information; and
transmitting a notification data packet containing at least a portion of the structured output to a terminal device of the user via the communication interface.
